(19) United States
(12) Patent Application Publication
(10) Pub. No.: US 2024/0350748 A1
(43) Pub. Date: Oct. 24, 2024

(54) NEEDLE SPACER AND SUBCUTANEOUS INJECTION SYSTEM INCLUDING SAME

(71) Applicant: Regeneron Pharmaceuticals, Inc., Tarrytown, NY (US)

(72) Inventors: Daniel HALBIG, Ballston Lake, NY (US); Matthew PAUSLEY, Gansevoort, NY (US); Thomas MADDEN, Ballston Lake, NY (US); Alexander ROUMANIDAKIS, Schenectady, NY (US); Sarah ELLIS, Albany, NY (US); Eric HOLOWKA, Glenmont, NY (US); Trevor LANGLEY, Rensselaer, NY (US); Jessica MASTROPIETRO, Schenectady, NY (US); Megan THOMPSON, Gansevoort, NY (US)

(21) Appl. No.: 18/639,051

(22) Filed: Apr. 18, 2024

Related U.S. Application Data
(60) Provisional application No. 63/460,189, filed on Apr. 18, 2023.

(51) Int. Cl. A61M 5/46; A61M 5/142; A61M 5/158; A61M 5/32

(52) U.S. Cl. CPC A61M 5/46; A61M 5/142; A61M 5/158; A61M 5/3287; A61M 2005/1586; A61M 2005/1588; A61M 2205/3327; A61M 2205/3331; A61M 2205/583; A61M 2209/088

(57) ABSTRACT

Needle spacers for use in administering subcutaneous injections, as well as subcutaneous injection systems including such needle spacers, are disclosed. In certain embodiments, the needle spacer comprises a first portion configured to engage a hub of a needle that comprises such a hub; and a second portion coupled to the first portion and extending distally therefrom, the second portion including a skin-contacting surface disposed on a distal end thereof, wherein the skin-contacting surface is configured to engage a surface of skin of a subject. The second portion is configured to maintain a predetermined distance between the hub of the needle and the surface of the skin, and to permit insertion of the needle to a depth of about 4 mm to about 8 mm below the surface of the skin during administration of a subcutaneous injection via the needle.

### CROSS REFERENCE TO RELATED APPLICATION

The present patent application claims priority to U.S. Provisional Patent Application No. 63/460,189, filed Apr. 18, 2023.

### FIELD OF THE DISCLOSURE

Various embodiments of the disclosure relate to systems for the administration of subcutaneous injections, as well as needle spacers for use in such systems to limit the injection depth of a needle to ensure that an injection is administered into the subcutaneous space.

### INTRODUCTION

Subcutaneous injections are used to administer medications and other fluids into the fatty tissue just below the surface of the skin. Medications suitable for administration via the subcutaneous route are varied and include, but are not limited to insulin, blood thinners such as heparin and enoxaparin (Lovenox® (a registered trademark of Sanofi Mature IP, Gentilly, FR)), fertility drugs/hormones, local anesthetics, drugs used in palliative care such as fentanyl and morphine, biologics such as etanercept (Enbrel® (a registered trademark of Immunex Corporation, Thousand Oaks, CA, US)), filgrastim (Neupogen® (a registered trademark of Amgen Inc., Thousand Oaks, CA, US)), and anakinra (Kineret® (a registered trademark of Swedish Orphan Biovitrum AB (PUBL), Stockholm, SE)), monoclonal antibodies, goserelin (Zoladex® (a registered trademark of AstraZeneca UK Limited, Södertälje, SE)), and other medications. Subcutaneous injections may also be used in other settings, including in clinical studies.

Subcutaneous injections require reliable insertion of a needle to a depth of about 4 mm to about 8 mm below the surface of the skin, and may be performed while maintaining a particular desired angle of the needle relative to the surface of the skin such as, e.g., a 90-degree angle. Additionally, it is desirable for the injection site to be readily visible to the clinician throughout the administration process to facilitate monitoring for, e.g., redness, leaking, swelling, and/or bruising at the injection site. These objectives may be difficult to reliably achieve when using a needle having a length of, e.g., about 13 mm or greater. Accordingly, there exists a need for a needle spacer and a system including such a needle spacer to facilitate reliable administration of subcutaneous injections, including achieving and maintaining a desired depth and insertion angle of the needle relative to the surface of the skin, for example about 4 mm to about 8 mm, and about 90-degrees, and supporting the needle at the desired depth and insertion angle against any tendency to bend or twist throughout the duration of the injection.

### SUMMARY

A first aspect of the disclosure provides a needle spacer comprising: a first portion configured to engage a hub of a needle, wherein the needle comprises such a hub; and a second portion coupled to the first portion and extending distally therefrom, the second portion including a skin-contacting surface disposed on a distal end thereof, wherein the skin-contacting surface is configured to engage a surface of skin of a subject, and wherein the second portion is configured to maintain a predetermined distance between the hub of the needle and the surface of the skin, and to permit insertion of the needle to a depth of about 4 mm to about 8 mm below the surface of the skin during administration of a subcutaneous injection via the needle.

Various embodiments of the needle spacer include one or more of the following features: wherein the needle spacer is configured to support the needle radially, axially, and laterally, such that the needle spacer maintains the needle at an approximately 90-degree angle with respect to the surface of the skin, and at a predetermined axial position with respect to the needle spacer while in use; wherein the needle spacer is configured to permit visualization of an injection site by a user during the administration of the subcutaneous injection; wherein the needle spacer is configured to permit qualitative or quantitative assessment of any leakage at the injection site during or after the administration of the subcutaneous injection; wherein one or both of the first portion or the second portion comprise a transparent material; wherein the second portion comprises an opening or a window through which the injection site is visible to the user during the administration of the subcutaneous injection; wherein the first portion of the needle spacer is configured to laterally receive the hub of the needle; wherein the first portion of the needle spacer is configured to axially receive the hub of the needle; wherein the skin-contacting surface further comprises a laterally extending wing or tab; wherein the laterally extending wing or tab further comprises a plurality of laterally extending wings or tabs; wherein the laterally extending wing or tab is configured to receive tape on a proximal face thereof, and wherein the tape adheres the laterally extending wing or tab to the skin; or wherein the skin-contacting surface comprises an adhesive disposed on a distal surface thereof.

Various embodiments of the needle spacer may additionally or alternatively include one or more of the following features: wherein the first portion of the needle spacer is further configured to engage a pressure sensor, and wherein the pressure sensor is fluidly coupled to the needle; wherein the first portion comprises a cavity therein open to a proximal end of the first portion, and the cavity comprises an interior profile configured to complement and to matingly receive a distal end of the needle hub; wherein a neck portion couples the first portion to the second portion, wherein the neck portion comprises an inner diameter that is greater than an outer diameter of the needle, and smaller than an outer diameter of the needle hub, such that the neck portion acts as a depth stop limiting movement of the needle hub in a distal direction, and wherein the second portion comprises a concave geometry that is open to a distal end of the needle spacer.

Various embodiments of the needle spacer may additionally or alternatively include one or more of the following features: wherein the first portion comprises a substantially annular shape having a first opening therein, the first opening being configured to laterally receive the hub of the needle; and the second portion comprises a leg member extending distally and radially outwardly relative to the first portion, and a base member coupled to the leg member, the base member comprising a substantially annular shape having a second opening therein, wherein the second opening is configured to laterally receive the needle therein, and is circumferentially aligned with the first opening, and a diameter of the base member is greater than a diameter of the first portion; wherein the leg member comprises a plurality of leg members, and each leg member of the plurality of leg members is circumferentially spaced about the first portion and the base member relative to each other leg member.

Various embodiments of the needle spacer may additionally or alternatively include one or more of the following features: an opening in a proximal end of the first portion, configured to receive the hub of the needle therein; and a plurality of snap arms disposed about the opening, wherein the plurality of snap arms are configured to collectively engage the hub of the needle; an opening in a proximal end of the first portion, configured to receive the hub of the needle therein; and a friction member disposed about a perimeter of the opening, wherein the friction member is configured to engage the hub of the needle, and the friction member is selected from a high friction coating and an O-ring; a plurality of circumferentially spaced arms coupled to the first portion, wherein each arm of the plurality of arms extends radially outwardly and distally relative to the first portion, and each arm includes a skin-contacting surface at a distal end thereof, and wherein the plurality of arms collectively form a domed shape, thereby maintaining the predetermined distance between the hub of the needle and the surface of the skin during the administration of the subcutaneous injection.

Various embodiments of the needle spacer may additionally or alternatively include one or more of the following features: wherein each arm in the plurality of arms comprises, at a distal end thereof, a curve about a circumference of the first portion; wherein the first portion comprises a hollow, substantially frustoconical portion tapering from a proximal end thereof to a distal end thereof, the first portion being configured to fit closely over a distal end of the hub of the needle, and wherein the second portion comprises a neck portion coupled to, and in communication with an interior of the hollow, substantially frustoconical portion at a frustum thereof, the neck portion having an inner diameter that is nominally larger than an outer diameter of the needle, such that the neck portion accommodates the needle therein, but does not accommodate the hub of the needle therein.

Various embodiments of the needle spacer may additionally or alternatively include one or more of the following features: wherein the first portion comprises a hollow cylinder having an opening in a distal end thereof; and an annular member disposed between the hollow cylinder and the needle hub.

Various embodiments of the needle spacer may additionally or alternatively include one or more of the following features: wherein the skin-contacting surface extends 360 degrees about the needle.

Various embodiments of the needle spacer may additionally or alternatively include one or more of the following features: wherein the first portion comprises two components having complementary geometries and being configured for snap fit engagement with one another about the hub of the needle.

Various embodiments of the needle spacer may additionally or alternatively include one or more of the following features: wherein the first portion is further configured to engage a pressure sensor disposed proximally of the hub of the needle, such that the hub of the needle is replaceable without disengaging the first portion from the pressure sensor; wherein the first portion comprises a tab configured to releasably engage a proximal end of the hub of the needle.

Various embodiments of the needle spacer may additionally or alternatively include one or more of the following features: wherein the first portion comprises a double-ended luer connector, having a male luer lock fitting and a female luer lock fitting on opposing ends, wherein the male luer lock fitting extends in a distal direction and engages the hub of the needle, and the female connection extends in a proximal direction, and wherein the double-ended luer connector, the needle hub, and the needle are in fluid communication with one another.

Various embodiments of the needle spacer may additionally or alternatively include one or more of the following features: wherein the first portion comprises a snap lock configured to laterally receive and engage the hub of the needle, and wherein the second portion extends further in a lateral direction than in a distal direction.

Various embodiments of the needle spacer may additionally or alternatively include one or more of the following features: wherein the first portion comprises a C-shaped spacer clip configured to engage the hub of the needle; wherein the second portion comprises a proximal shoulder, against which the C-shaped spacer clip is configured to bear in use; wherein the first portion comprises external threads.

Various embodiments of the needle spacer may additionally or alternatively include one or more of the following features: wherein the second portion comprises a sleeve disposed over the first portion, the sleeve having internal threads on an inner surface thereof, and an internal shoulder disposed distally relative to the internal threads, wherein the external threads are configured to threadably engage the internal threads to cause translation of the first portion relative to the second portion; and as the first portion advances distally relative to the second portion, the internal shoulder biases a distal end of the first portion against a radially outer surface of the needle.

Various embodiments of the needle spacer may additionally or alternatively include one or more of the following features: wherein the hub of the needle comprises a female luer fitting, and the first portion of the needle spacer is configured to engage a male luer fitting that is coupled to the female luer fitting.

Various embodiments of the needle spacer may additionally or alternatively include one or more of the following features: wherein the first portion comprises a housing having a cavity therein, and a laterally accessible opening to the cavity, and the laterally accessible opening and the cavity are adapted to matingly receive the male luer fitting; and wherein the window in the second portion is in fluid communication with the laterally accessible opening in the first portion, such that as the male luer fitting is laterally received in the cavity, the window is adapted to laterally receive the needle and a needle shield disposed over the needle.

Various embodiments of the needle spacer may additionally or alternatively include one or more of the following features: a plurality of snap fit features disposed on each side of the laterally accessible opening, wherein the plurality of snap fit features are adapted to flex radially outwardly to allow insertion of the male luer fitting, and to retain the male luer fitting within the cavity; and a relief cut in a side wall of the housing, disposed opposite the laterally accessible opening, wherein the relief cut is adapted to receive a rib on the male luer fitting.

A second aspect of the disclosure provides a system comprising: a needle coupled to a hub and configured for subcutaneous injection into skin of a subject; tubing coupled to, and in fluid communication with the hub and the needle; and a needle spacer disposed between, and configured to maintain a predetermined distance between, the hub of the needle and the skin of the subject. The needle spacer comprises a first portion configured to engage the hub of the needle; and a second portion coupled to the first portion and extending distally therefrom, the second portion including a skin-contacting surface disposed on a distal end thereof. The skin-contacting surface is configured to engage the surface of the skin, and the second portion is configured to permit insertion of the needle to a depth of about 4 mm to about 8 mm into the skin during administration of a subcutaneous injection via the needle.

Various embodiments of the needle spacer may additionally or alternatively include one or more of the following features: a syringe fluidly coupled to a proximal end of the tubing; and an infusion pump configured to administer a fluid via subcutaneous injection, wherein the fluid is selected from a medicament, a solution, a suspension, saline, or hyaluronic acid; wherein the needle has a length of about 13 mm or greater; wherein a distal end of the tubing is coupled to the needle hub by complementary luer fittings, wherein the complementary luer fittings are luer lock fittings, wherein the needle spacer is configured to permit unimpeded manipulation of the luer fittings in use, and wherein a pressure sensor is provided in fluid connection with the needle, the hub, and the tubing, wherein the first portion of the needle spacer is configured to engage the pressure sensor.

These and other aspects, advantages, and salient features of the disclosure will become apparent from the following detailed description, which, when taken in conjunction with the annexed drawings, disclose embodiments of the invention.

### BRIEF DESCRIPTION OF THE DRAWINGS

The accompanying drawings, which are incorporated into and constitute a part of this specification, illustrate various exemplary embodiments and, together with the description, serve to explain the principles of the disclosed embodiments. The drawings show different aspects of the present disclosure. Where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

The embodiments described herein are not limited to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIGS. 1A-1C show perspective views of various systems in accordance with embodiments of the disclosure.

FIGS. 2A, 2B, and 2C show a cross sectional view, a cross sectional view along line A-A (shown in FIG. 2A), and a bottom view, respectively, of a needle spacer in accordance with an embodiment of the disclosure.

FIG. 3 shows a perspective view of a needle spacer in accordance with an embodiment of the disclosure.

FIG. 4A shows a cross sectional view of a needle spacer in accordance with an embodiment of the disclosure.

FIG. 4B shows a cross sectional view of a portion of a needle spacer in accordance with an embodiment of the disclosure.

FIG. 4C shows a top view of the needle spacer of FIG. 4A, in accordance with an embodiment of the disclosure.

FIG. 4D shows a luer removal tool for removing a needle from the needle spacer of FIGS. 4A-4C in accordance with embodiments of the disclosure.

FIGS. 5A and 5B show cross sectional and top views, respectively, of a needle spacer in accordance with embodiments of the disclosure.

FIGS. 6A, 6B, and 6C show front, cross sectional, and bottom views, respectively, of a needle spacer in accordance with embodiments of the disclosure.

FIGS. 7A, 7B, and 7C illustrate various views of a needle spacer in accordance with embodiments of the disclosure, with FIGS. 7A and 7B providing cross sectional views, and FIG. 7C providing a top view.

FIGS. 8A, 8B, 8C, 8D, and 8E illustrate various views of a needle spacer in accordance with embodiments of the disclosure, with FIG. 8A providing a side view of a needle including a hub, FIG. 8B providing a perspective view of a portion of a needle spacer, FIG. 8C providing a partial side view of a needle spacer, FIG. 8D providing a cross sectional view of a needle spacer, and FIG. 8E providing an exploded top view of a needle spacer.

FIG. 9 shows a front view of a needle spacer in accordance with embodiments of the disclosure.

FIGS. 10A and 10B show a front view and a perspective view, respectively, of a needle spacer in accordance with embodiments of the disclosure.

FIG. 11 shows a cross sectional view of a needle spacer in accordance with embodiments of the disclosure.

FIGS. 12A, 12B, and 12C provide side, top, and cross-sectional views of a needle spacer in accordance with embodiments of the disclosure.

FIGS. 13A, 13B, 13C, and 13D illustrate various views of a needle spacer in accordance with embodiments of the disclosure, with FIGS. 13A and 13D providing cross sectional views, FIG. 13B providing a top view of the spacer clip of FIG. 13A, and FIG. 13C providing a top view of a portion of the needle spacer of FIG. 13A.

FIG. 14 shows a cross sectional view of a needle spacer in accordance with an embodiment of the disclosure.

FIGS. 15A and 15B show front and perspective views, respectively, of a needle spacer in accordance with embodiments of the disclosure.

FIGS. 16A, 16B, 16C, and 16D show side, bottom, side, and top perspective views, respectively, of a needle spacer in accordance with embodiments of the disclosure.

FIG. 16E shows a cross sectional view of a portion of needle spacer, taken along line A-A as shown in FIG. 16C, in accordance with embodiments of the disclosure.

FIGS. 17A, 17B, 17C, 17D, and 17E illustrate side perspective views of steps in the assembly of the needle spacer of FIGS. 16A-16E in accordance with embodiments of the disclosure.

It is noted that the drawings of the disclosure are not necessarily to scale.

### DETAILED DESCRIPTION

Embodiments of the present disclosure relate to systems for the administration of subcutaneous injections, as well as needle spacers for use in such systems to limit the injection depth of the needle to ensure that the injection is administered into the subcutaneous space.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

The term “distal end” or any variation thereof, refers to the portion of a device farthest from an operator of the device during an injection operation. For example, the distal end of a syringe would be the needle end of the syringe, and the distal end of a needle would be the sharp tip. Conversely, the term “proximal end,” or any variation thereof, refers to the portion of the device closest to the operator of the device during an injection operation. For example, the proximal end of a syringe would be the plunger end of the syringe, and the proximal end of a needle would be the end coupled to a needle hub. Further, as used herein, the terms “about,” “substantially,” and “approximately” generally mean ±10% of the indicated value.

As used herein, the term “subject” refers to an animal on whom the subcutaneous injection is to be performed, i.e., to whom the subcutaneous injection may be administered. The animal may be a mammal, and may more particularly be a human, or may be a domesticated animal such as a dog, cat, ferret, hamster, rabbit, mouse, rat, cattle, sheep, pig, goat, horse, donkey, camel, and so on. The terms “clinician” and “user” are used interchangeably to refer to the individual performing the administration of the subcutaneous injection on the subject as described herein.

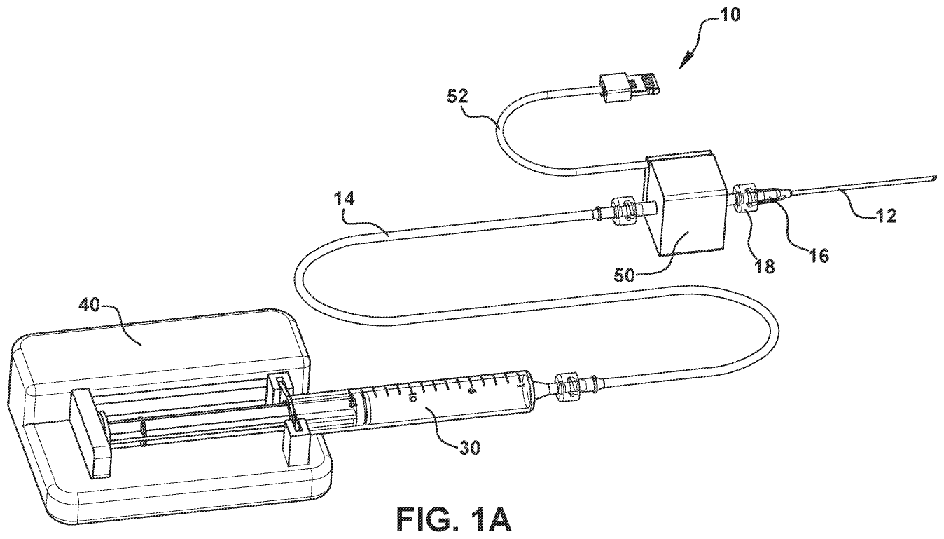

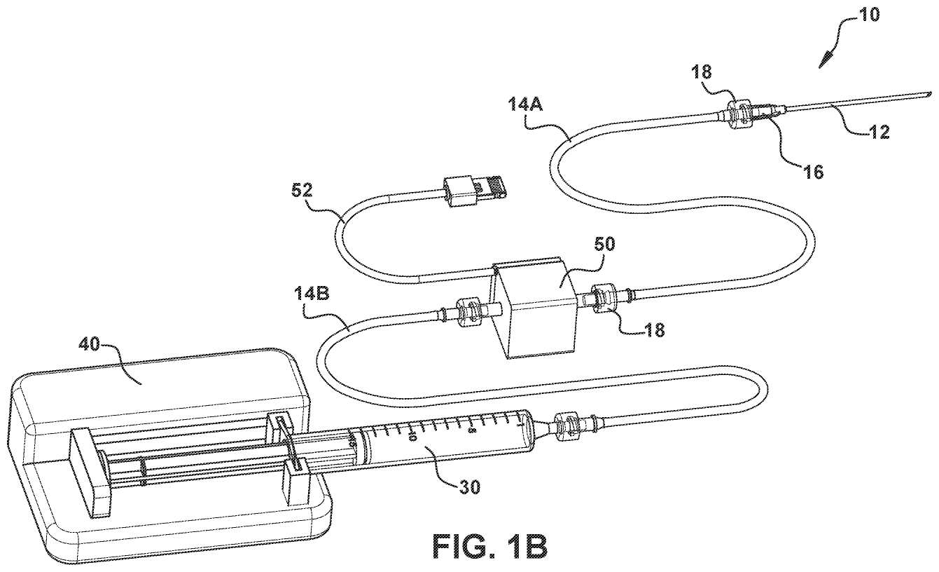

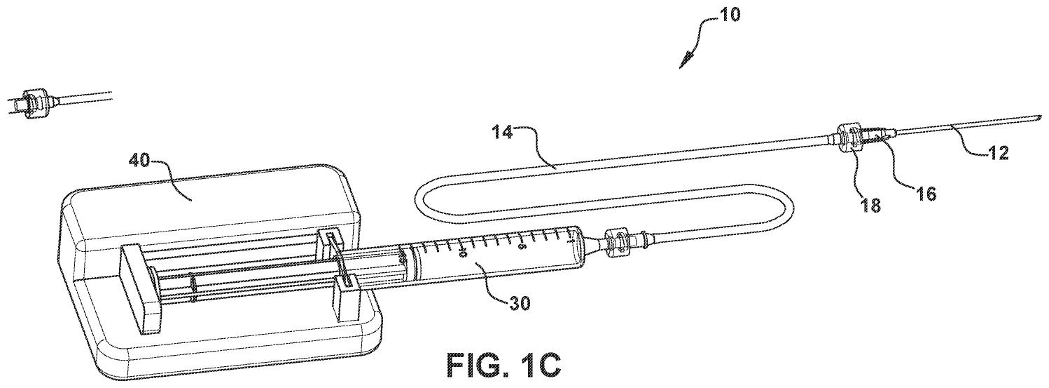

As indicated above, FIGS. 1A-1C illustrate certain aspects of the disclosure that provide systems for administering subcutaneous injections to a subject. As shown in FIGS. 1A-1C, the system 10 comprises a needle 12 that includes a hub 16 coupled to a proximal end of the needle 12. The needle 12 is configured for subcutaneous injection into the skin of a subject, e.g., at an angle of 90-degrees relative to the surface of the skin. The needle may have a length of, e.g., 13 mm, 14 mm, 15 mm, 16 mm, or greater, and may be of any suitable diameter, e.g., 19 gauge, 20 gauge, 21 gauge, 22 gauge, 23 gauge, 24 gauge, 25 gauge, 26 gauge, or 27 gauge. In certain embodiments, the hub 16 may include a female luer connection. The system 10 further comprises tubing 14 directly or indirectly coupled to, and in fluid communication with the hub 16 and the needle 12. The tubing 14 may further be fluidly coupled, e.g., at a proximal end thereof, either directly or indirectly to a syringe 30.

In certain embodiments, the system 10 may further include a pressure sensor 50 in fluid connection with the needle 12, the hub 16, tubing 14, and the syringe 30. For example, the pressure sensor 50 may be a single use luer pressure sensor such as made by PendoTech, Inc., Princeton, NJ. The pressure sensor 50 may be configured to collect in-line pressure and back pressure data, and communicate such data to a pressure monitor (not shown), e.g. via cable 52 (FIGS. 1A-1B).

In one exemplary system, shown in FIG. 1A, the system 10 may include a needle 12 having a hub 16, which may include a female luer connection. The hub 16 may be coupled to a pressure sensor 50 at a distal end thereof. For example, the pressure sensor 50 may include a male luer connection (alternatively referred to herein as a luer “fitting” or “adapter”) 18 at a distal end thereof that provides a complementary fit with the female luer connection of the hub 16. The female and male luer connectors 16 and 18, respectively, may be complementary luer fittings which may be, e.g., female and male slip luers or female and male luer lock fittings which include a twist-lock mechanism to hold the luer connectors 16, 18 together. The pressure sensor 50 may be may coupled at a proximal end thereof to tubing 14, which in turn may be coupled at a proximal end thereof to the syringe 30. Each of the foregoing connections may further comprise complementary female and male luer connections. For example, the syringe 30 may include a male luer connector which may engage a female luer connector at a proximal end of the tubing 14, and a distal end of the tubing 14 may include a male luer connector which may engage a female luer connector disposed on a proximal end of the pressure sensor 50.

In another exemplary embodiment, shown in FIG. 1B, the system 10 may include a needle 12 having a hub 16, which may include a female luer connection. The hub 16 may be coupled to tubing 14A, which may include male luer connections 18 disposed at both of the distal and proximal ends of the tubing 14A. Tubing 14A may therefore act as a luer adapter, facilitating coupling of the hub 16 to a pressure sensor 50 having a female luer connection at a distal end thereof. The pressure sensor 50 may be coupled at a proximal end thereof to tubing 14B, which may in turn couple at a proximal end thereof to the syringe 30. Each of the foregoing connections may further include complementary female and male luer connections. For example, the syringe 30 may include a male luer connector which may engage a female luer connector at a proximal end of the tubing 14B, and a distal end of the tubing 14B may include a male luer connector which may engage a female luer connector disposed on a proximal end of the pressure sensor 50.

In a further exemplary embodiment, shown in FIG. 1C, the hub 16 may be coupled to a distal end of tubing 14, e.g., by complementary female and male luer connectors, respectively. The tubing 14 may in turn be directly coupled at a proximal end thereof to the syringe 30 by complementary female and male luer connectors, respectively.

In each of the foregoing embodiments of FIGS. 1A-1C, fluid held in the reservoir of the syringe 30 may be administered using an infusion pump 40, in which a movable piston controls fluid delivery. As described herein, the fluid is delivered via a subcutaneous injection route, using the infusion pump 40, syringe 30, tubing 14, pressure sensor 50 (if present, such as in FIGS. 1A-1B), hub 16, and needle 12. Fluids of various types may be administered using the system 10 described herein, as will be understood by the skilled clinician. Exemplary fluids to be administered include medicaments, solutions, suspensions, or saline. The system 10 may be used to administer fluids having a relatively high viscosity such as, e.g., hyaluronic acid. Suitable medicaments may include, e.g., insulin, blood thinners such as heparin and enoxaparin (Lovenox®), fertility drugs/hormones, local anesthetics, drugs used in palliative care such as fentanyl and morphine, biologics such as etanercept (Enbrel®), filgrastim (Neupogen®), and anakinra (Kineret®), monoclonal antibodies, goserelin (Zoladex®), and other medications approved for administration via subcutaneous injection or in clinical or pre-clinical development.

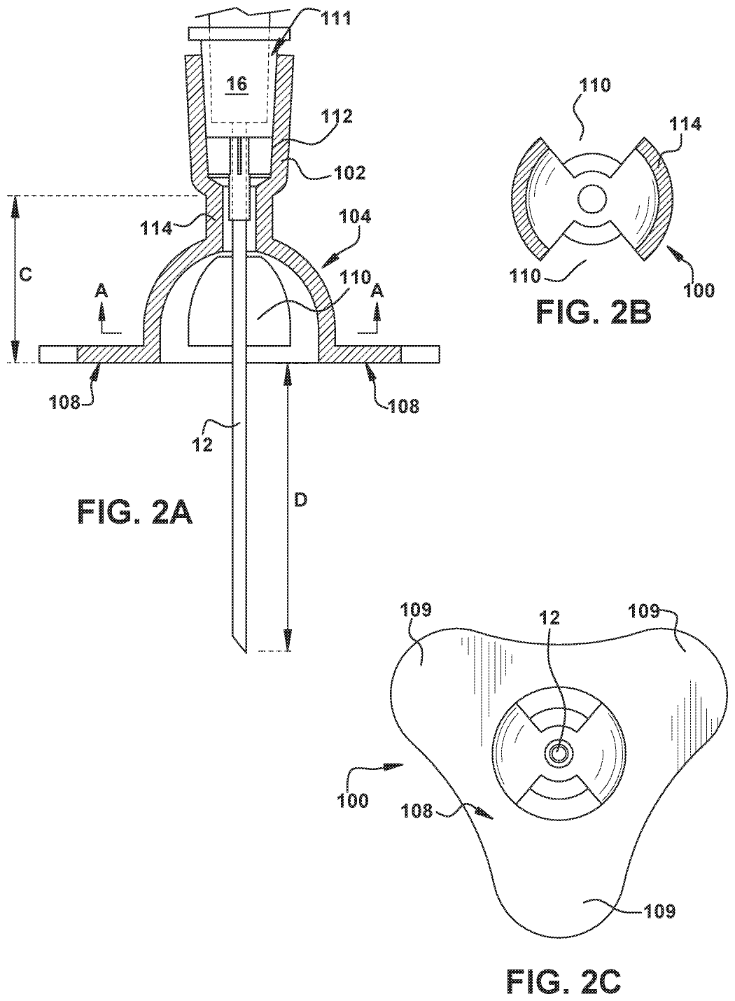

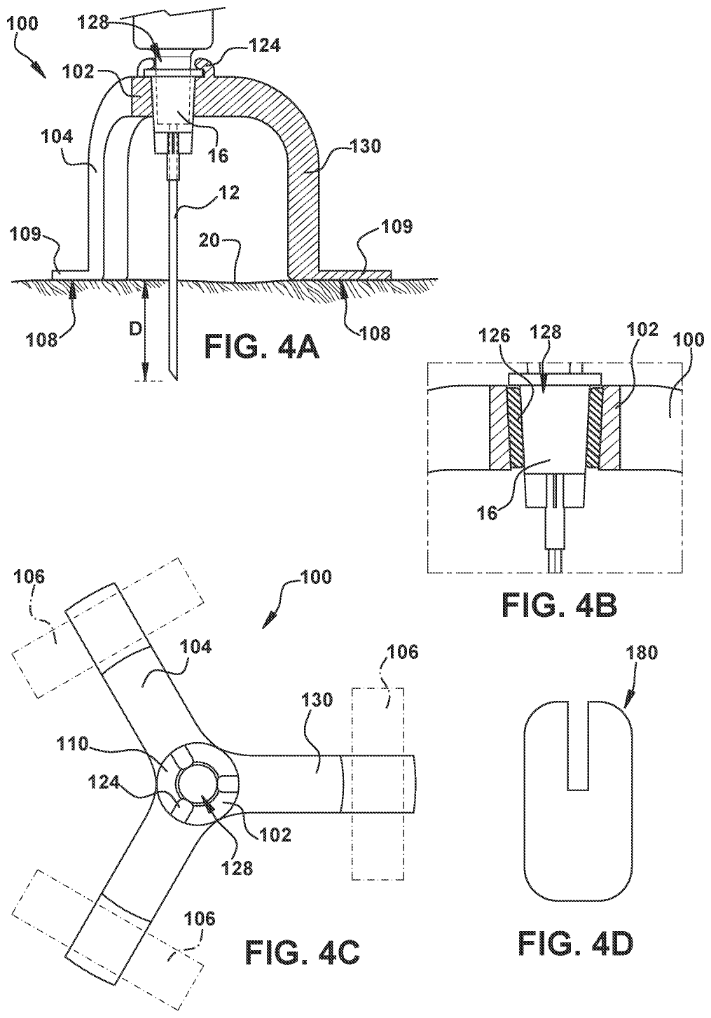

With reference now to FIGS. 2A through 16E, a needle spacer 100 is provided, which may be used in connection with the elements of system 10 shown in FIGS. 1A-1C and described above. The needle spacer 100 is disposed between the hub 16 of the needle 12 (FIGS. 1A-1C), and the skin 20 of the subject (FIGS. 4A, 5A, 6A, 7A, 13A, 13D), and is configured to maintain a predetermined distance between the hub 16 of the needle 12 and the skin 20 and angle of the needle relative to the surface of the skin 20 as shown in, e.g., FIGS. 2A, 4A-4B, 5A, 6B, 7A-7B, 8A, 8D, 9, 11, 12A, and 13A. Accordingly, the needle spacer provides the ability to reliably insert the needle 12 into the skin 20 to a predetermined and desired depth.

The needle spacer 100 comprises a first portion 102 that is configured to engage the hub 16 of the needle 12, as well as a second portion 104 that is coupled to the first portion 102. The second portion 104 extends distally from the point at which it is coupled to the first portion 102. The second portion 104 includes a skin-contacting surface 108 disposed on a distal end thereof, wherein the skin-contacting surface 108 is configured to engage the surface of the skin 20.

Through interactions which are described further hereinbelow, the second portion 104 is configured to act as a depth stop for the needle 12 and hub 16. The needle spacer 100, including the second portion 104, is configured to permit insertion of the needle 12 to a depth D (labeled in FIGS. 2A, 4A, 5A, 7A, 13D, and 15A), which may be about 4 mm to about 8 mm, about 6 mm to about 8 mm, about 6 mm, about 7 mm, or about 8 mm into the skin 20 during administration of a subcutaneous injection. The needle spacer 100, including the second portion 104, is further configured to limit or prevent entirely the insertion of the needle 12 to a depth beyond the depth D. Accordingly, the needle spacer 100 provides the ability to reliably insert the needle 12 into the skin 20 to the desired depth.

In addition, the needle spacer 100 is configured to support the needle 12 radially, axially, and laterally, either directly or indirectly via engagement with the hub 16 of the needle 12. As a result, the needle spacer 100 may be adapted to maintain the needle 12 at an approximately 90-degree angle with respect to the surface of the skin 20, and at a predetermined axial position with respect to the needle spacer 100 while in use. This eliminates or reduces the need for a human clinician to hold the needle 12 during use.

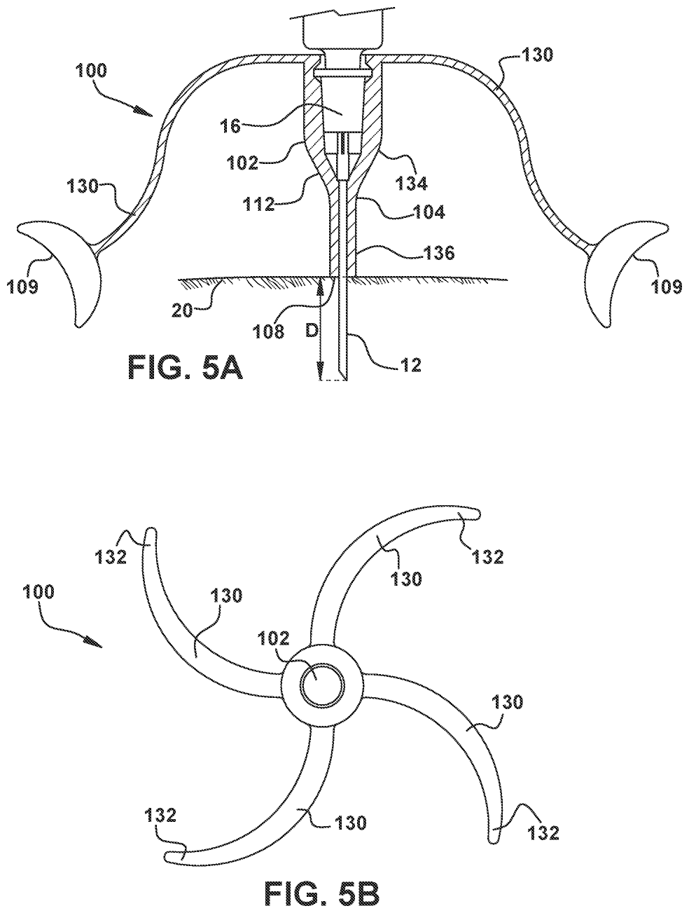

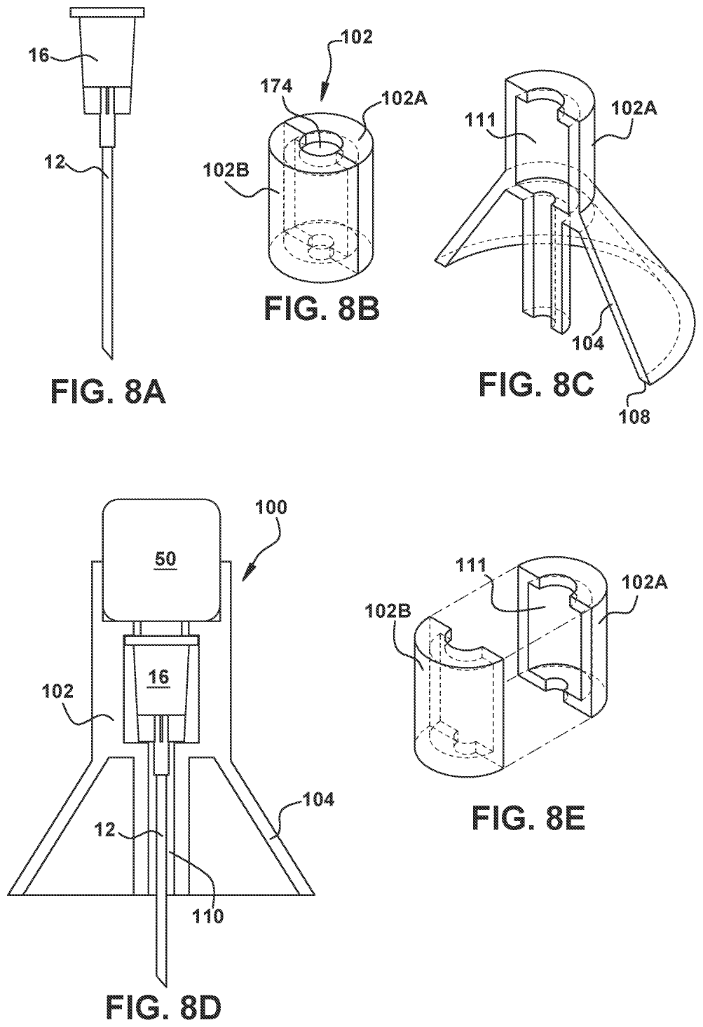

The needle spacer 100 is further configured to permit visualization of an injection site by a clinician during the entire subcutaneous injection administration process. To provide this visibility, in certain embodiments, one or both of the first portion 102 or the second portion 104 are made of a transparent material (e.g., FIGS. 5A-5B, 8A-8E). The transparent material may be, e.g., plastic. The needle spacer 100 may be formed by any known method such as, e.g., injection molding or additive manufacturing (3D printing). In other embodiments, the second portion 104 may include an opening or a window 110 (e.g., FIGS. 2A-2B, 3, 4C, 9, 10A-10B, 11, and 16A) through which the injection site is visible to the clinician during the administration of the subcutaneous injection.

The foregoing features permit a clinician to perform qualitative or quantitative assessment of any leakage at the injection site during or after the administration of the subcutaneous injection. For example, the needle spacer 100, and particularly openings or windows 110 may facilitate access for the user to, in the event of leakage at the injection site, absorb all leaked fluid volume to be weighed. The needle spacer 100, and particularly openings or windows 110 and transparent material from which the needle spacer 100 is constructed may also facilitate access for the user to monitor the injection site for redness, swelling, bruising, and other adverse events.

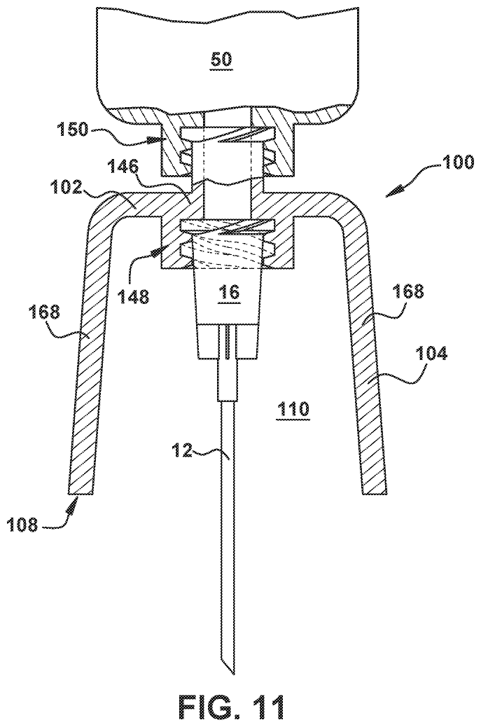

The material used to fabricate the needle spacer 100 may in some embodiments be compatible with sterilization. Sterilization may or may not be necessary in clinical applications, depending on whether the needle spacer will come into contact with fluids as in, e.g., the embodiment shown in FIG. 11.

The needle spacer 100 may further be configured to permit unfettered or unimpeded access to and manipulation of any luer connectors in use in the system 10, e.g., coupling the hub 16 of the needle 12 to the tubing 14 and/or pressure sensor 50.

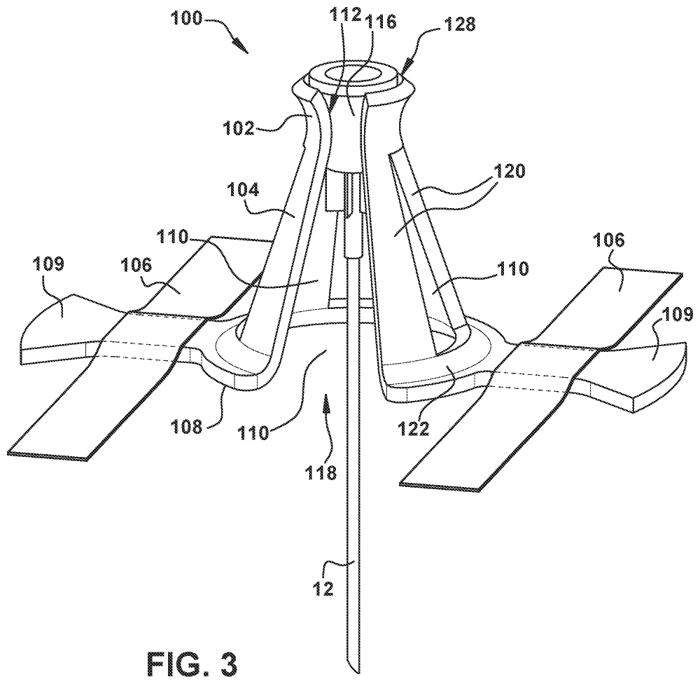

In certain embodiments, such as those shown in FIGS. 3, 6A-6C, 8A-8E, 9, 10A-10B, 12A-12C, 13A-13C, 15A-15B, and 16A-16E, the first portion 102 of the needle spacer 100 is configured to laterally receive the hub 16 of the needle 12. In such embodiments, the first portion 102 of the needle spacer includes an opening for insertion in a direction perpendicular to the longitudinal axis of the needle 12.

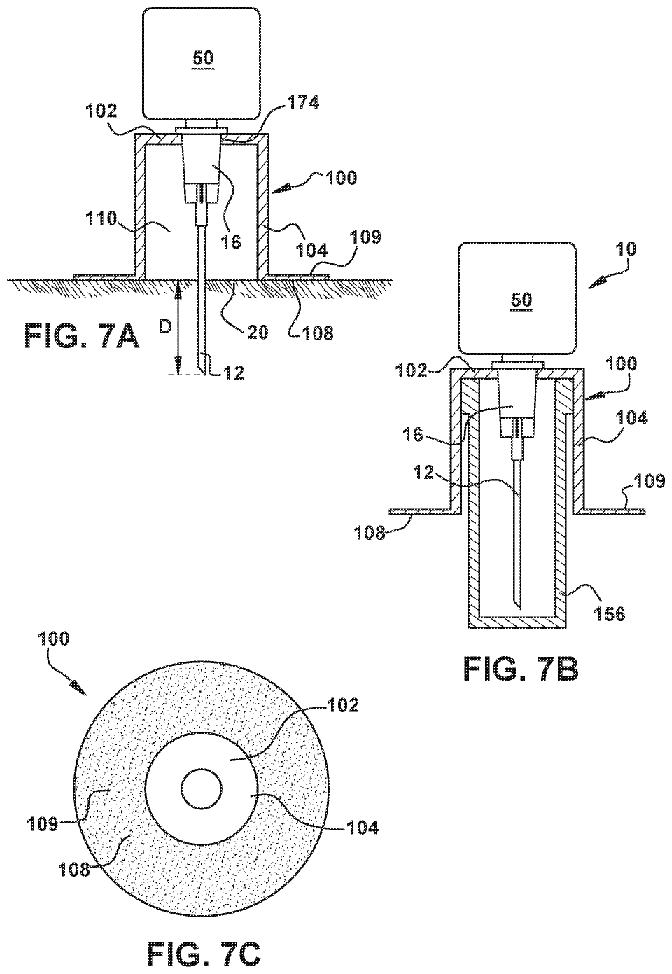

In certain embodiments, such as those shown in FIGS. 2A-2C, 4A-4D, 5A-5B, 7A-7C, 11, and 14, the first portion 102 of the needle spacer 100 is configured to axially receive the hub 16 of the needle 12. In such embodiments, e.g., that of FIGS. 2A-2C, 4A-4C, and 5A, the first portion 102 of the needle spacer includes an opening configured to receive a needle 12 and/or hub 16 that is inserted in a direction parallel to and/or coaxial with the longitudinal axis of the needle 12. In certain embodiments, e.g., that of FIGS. 5A-5B, the needle may be axially inserted in a distal direction, distal tip first, through a proximal end of the first portion 102. In other embodiments, e.g., that of FIGS. 7A-7C, the needle 12 may be axially inserted in a proximal direction, in which the hub 16 is inserted first, into the distal end of the first portion 102. This may entail inserting the hub 16 through second portion 104 to reach first portion 102, and may permit first portion 102 of the needle spacer 100 to engage the hub 16 of the needle 12 while a needle shield 156 remains in place, covering the sharp, distal tip of the needle 12.

In certain embodiments, such as for example those shown in FIGS. 2A-2C, 5A, 7A-7C, 14, and 16A, the skin-contacting surface 108 may include a unitary, continuous skin-contacting surface 108. In other embodiments, such as for example those shown in FIGS. 4A-4C, 6C, 8A-8E, 9, 10A-10B, and 11 the skin-contacting surface 108 may include a plurality of skin-contacting surfaces 108 disposed on the distal end of the second portion 104. In some embodiments, regardless of the number of skin-contacting surfaces 108 present, one or more of the skin-contacting surfaces 108 may further comprise a laterally extending wing or tab 109. Wings or tabs 109 provide additional surface area over which the needle spacer 100, and particularly second portion 104 may contact the skin 20, which may contribute additional stability to the needle spacer 100 while in use. In various embodiments, one, two, three, four, or more wings or tabs 109 may be present. For example, FIG. 7C illustrates a single wing or tab 109;

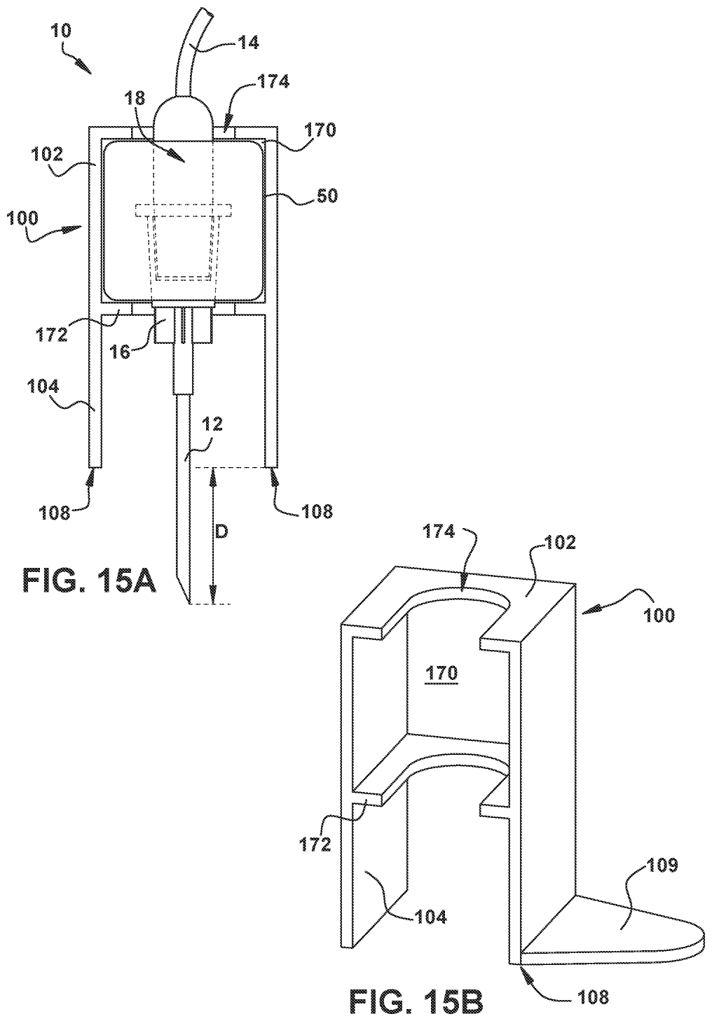

FIGS. 3, 6C, 12B-12C, and 13B-13C illustrate two wings 109; and FIGS. 2C, 4A, and 4C illustrate three wings 109. FIG. 15B illustrates a single wing or tab 109, however additional wings or tabs 109 may be used in conjunction with this or any embodiment as will be apparent to the skilled individual. Laterally extending wings or tabs 109 may be adapted to receive tape 106 over a proximal face thereof, as shown in, e.g., FIGS. 3, 4C, 12B, and 13C. The tape may be used to adhere the needle spacer 100 to the skin 20 of the subject. In other embodiments, the skin-contacting surface 108, which may include one or more tabs or wings 109, may include an adhesive disposed on a distal surface thereof. The adhesive may be single use or reusable, and may be self-sticking, as shown in FIG. 7C. In still further embodiments, any needle spacer described herein may be used in the absence of any tape or adhesive.

As discussed above, certain embodiments of the system 10 include a pressure sensor 50 (shown in, e.g., FIGS. 1A-1B, 7A-7B, 9, 10A, 12A, and 15A) in fluid connection with the needle 12. In certain embodiments, the hub 16 of the needle 12 may be coupled to the pressure sensor 50, and the first portion 102 of the needle spacer 100 may be configured to engage the pressure sensor 50 in addition to the hub 16 of the needle 12. The geometry of the first portion 102 may be selected to complement that of the pressure sensor, allowing for, e.g., snap fit engagement between the first portion 102 of the needle spacer 100 and the pressure sensor 50.

Turning next to the specific embodiments of the needle spacer 100, the foregoing advantages and features may be combined in a number of ways, including but not limited to those discussed herein and illustrated in the appended drawings.

FIGS. 2A-2C illustrate a needle spacer 100 according to one embodiment of the disclosure. As shown, the first portion 102 is configured to engage a hub 16 of a needle 12 that comprises such a hub 16. In particular, the first portion 102 may take the form of a collar disposed about a circumference of the hub 16, and may comprise a cavity 111 therein, which may be open to a proximal end of the first portion 102. The needle 12 including hub 16 may be axially inserted into the cavity 111 in a distal direction, with the tip of needle 12 being inserted first. The cavity 111 may include an interior profile 112 that has a geometry that is complementary to that of the hub 16 of the needle 12, such that the hub 16 may be matingly received within the cavity 111. A close fit may be provided between the hub 16 and the cavity 111, which may include a snap fit engagement.

A neck portion 114 may be coupled to the distal end of the first portion 102, and in fluid connection therewith. The neck portion 114 may have an inner diameter that is greater than the outer diameter of the needle 12, such that the neck portion 114 accommodates the needle 12 therein. However, the inner diameter of the neck portion 114 may be smaller than the outer diameter of the distal end of the needle hub 16. Accordingly, the neck portion acts as a depth stop limiting movement of the needle hub 16 in a distal direction. As shown, the outer diameter of neck portion 114 is smaller than the outer diameter of the first portion 102, although this need not be the case.

A second portion 104 is coupled to the distal end of the neck portion 114 and extends distally therefrom. The second portion 104 may include a skin-contacting surface 108 disposed on a distal end thereof. The second portion 104 may further take the form of a dome or have a concave geometry that is open to the distal end of the needle spacer 100, such that the skin-contacting surface 108 includes a perimeter of the domed or concave shaped second portion 104. The skin contacting surface 108 may further include one or more wings or tabs 109, such as shown in FIG. 2C, in which three tabs 109 extend radially outwardly from the center of the skin contacting surface 108. As illustrated, the tabs 109 shown in FIG. 2C are substantially lobe shaped, although any other configuration known in the art may be substituted. In other embodiments, one, two, four, or more tabs 109 may be used. Tabs 109 increase the contacting surface area between the needle spacer 100 and the skin 20, thereby increasing stability. Tabs 109 also provide points of contact wherein tape may be applied over the tab 109 and the subject's skin to adhere the needle spacer 100 to the skin.

The second portion 104 is configured to maintain a predetermined distance between the hub 16 of the needle 12 and the surface of the skin 20, and to permit insertion of the needle 12 to a depth D of about 4 mm to about 8 mm, e.g., about 6 mm to about 8 mm, about 6 mm, about 7 mm, or about 8 mm below the surface of the skin 20 during administration of a subcutaneous injection. In particular, with reference to FIG. 2A, the depth D is a function of the needle length and the distance C, which is the distance from the depth stop at the transition from the first portion 102 to the neck portion 114, to the skin-contacting surface 108. More particularly, the depth D may be the difference between the length of needle 12 in millimeters, and the distance C, which is the distance in millimeters from the depth stop at the transition from the first portion 102 to the neck portion 114, to the skin-contacting surface 108.

In part through the engagement of the first portion 102 with the hub 16, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use. The needle spacer 100 is further configured to permit visualization of the injection site by a user during the administration of the subcutaneous injection, e.g., through windows 110, as shown in FIG. 2B. Windows 110 may be, e.g., openings in the material from which the second portion 104 is constructed. Windows 110 further provide access to the injection site to permit qualitative or quantitative assessment of any leakage at the injection site during or after the administration of the subcutaneous injection.

FIG. 3 illustrates a needle spacer 100 according to another embodiment of the disclosure. As shown, the first portion 102 is configured to engage a hub 16 of a needle 12 that comprises such a hub 16. The first portion 102 may have a substantially annular shape which includes a first opening 116 or break in the annulus. It is noted that the terms “annular” and “circumference” are used to describe the embodiment shown in FIG. 3, although a perfectly annular or circular profile is not required; a slightly oblong or other shape may also be used, such as, e.g., a square, hexagon, octagon, etc. Returning to FIG. 3, the first opening 116 may be configured to laterally receive the hub 16 of the needle 12. In some embodiments, snap fit engagement may be provided between the hub 16 and the first opening 116.

A second portion 104 may be coupled to the first portion 102 and extend distally therefrom. As shown in FIG. 3, the second portion 104 may include one, two, three, four, or more leg members 120 extending distally and radially outwardly relative to the first portion 102. Four leg members 120 are shown in FIG. 3. Where a plurality of leg members 120 are included, each leg member 120 may be circumferentially spaced, e.g., evenly or approximately evenly circumferentially spaced about the first and second portions 102, 104 with respect to each other leg member 120. The second portion 104 may further include a base member 122 coupled to the leg member(s) 120 at a distal end thereof. The base member 122 may have an annular cross-sectional shape or another alternative geometry such as, e.g., a square, hexagon, octagon, etc. The base member 122 may have a perimeter, e.g., a circumference, that is greater than that of the first portion 102, such that the second portion 104 is flared in shape. The bottom, or distal-facing surface of the base member 122 may include a skin-contacting surface 108. The base member 122 may further include a second opening 118 therein, that is configured to laterally receive the needle therein. The first and second openings 116, 118 may be circumferentially aligned with one another, so that the needle 12 including hub 16 may be laterally inserted as a unit through first and second openings 116, 118. The opening 118 may further be wide enough to laterally accommodate a needle shield (such as needle shield 156 shown in FIG. 7B) therethrough, so that the needle shield may remain in place shielding the tip of the needle 12 during connection to the needle spacer 100, and may be removed only after the needle hub 16 and needle spacer 100 are engaged.

The base member 122 may further include one or more tabs or wings 109 extending laterally therefrom. FIG. 3 illustrates two tabs 109 extending from base member 122. In other embodiments, one, three, four, or more tabs 109 may be used. Tabs 109 increase the contact surface area between the needle spacer 100 and the skin 20, thereby increasing stability. Tabs 109 also provide points of contact over which tape 106 may be applied, to adhere the needle spacer 100 to the subject's skin.

The second portion 104, which may include the leg(s) 120 and the base member 122, is configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. This may be achieved by selecting a length for the leg member(s) 120 that will result in the needle 12 extending beyond the skin-contacting surface 108 by the desired depth. In part due to the tight fit, which may be a snap fit between the hub 16 and the first portion 102, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection. As shown, leg members 120 are spaced from one another sufficiently to provide easy access for a clinician to visualize the injection site from most points. This ease of access also permits qualitative or quantitative assessment of any leakage at the injection site during or after the administration of the subcutaneous injection.

FIGS. 4A-4C illustrate a needle spacer 100 according to another embodiment of the disclosure, together with a luer removal tool 180 in FIG. 4D for associated use. As shown, the first portion 102 is configured to engage a hub 16 of a needle 12 that comprises such a hub 16. An opening 128 may be provided in a proximal end of the first portion 102, the opening 128 being configured to axially receive and engage the hub 16 of the needle 12 therein. In particular, the needle 12 may be inserted, tip first, into the opening 128 at the proximal end of the first portion 102. In one embodiment (shown in FIGS. 4A and 4C), a plurality of snap arms 124 may be disposed about the opening 128, wherein the plurality of snap arms 124 are configured to collectively engage the hub 16 of the needle 12. As shown (FIG. 4C), three snap arms 124 may be provided, although other numbers of snap arms may also be used, e.g., two, four, or more snap arms. In another embodiment, shown in FIG. 4B, a friction member 126 may be disposed about a perimeter of the opening 128 in lieu of the snap arms 124. The friction member 126, which may be, e.g., a high friction surface coating such as thermoplastic elastomer (TPE) or an O-ring made of any suitable material such as rubber, may be configured to engage the hub 16 of the needle 12.

A second portion 104 is coupled to the first portion 102 and may include a plurality of circumferentially spaced arms 130 coupled to the first portion 102, each arm 130 extending radially outwardly and distally relative to the first portion 102. The plurality of arms 130 may be individually arched, and may collectively form a domed shape, thereby maintaining the predetermined distance between the hub 16 of the needle 12 and the surface of the skin 20 during the administration of the subcutaneous injection. FIG. 4C illustrates three arms 130, although more or fewer arms 130 may be included, e.g., two, four, or more arms.

Each arm 130 may include a skin-contacting surface 108 at a distal end thereof. In order to provide greater stability and opportunity for adhesion to the skin 20, the skin-contacting surface 108 on each arm 130 may further include a wing or tab 109, which provides increased surface area over which tape 106 or other adhesive may be applied.

The second portion 104 is configured to maintain a predetermined distance between the hub 16 of the needle 12 and the surface of the skin 20, and to permit insertion of the needle 12 to a depth D of about 4 mm to about 8 mm, e.g., about 6 mm to about 8 mm, about 6 mm, about 7 mm, or about 8 mm below the surface of the skin 20 during administration of a subcutaneous injection. This may be achieved by selecting the height of the dome formed by the plurality of arms 130 (FIG. 4A), relative to the length of the needle 12 being used. The needle spacer 100 is configured to support the needle 12 radially, axially, and laterally, for example through its fit with first portion 102, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection, for example, through windows 110 (FIGS. 4A, 4C). Windows 110 may comprise open spaces between each of the plurality of arms 130, which may permit qualitative or quantitative assessment of any leakage at the injection site during or after the administration of the subcutaneous injection, as well as monitoring for swelling, bruising, redness, or other adverse events.

In use, the needle spacer 100 may first be adhered to the skin 20, e.g., using tape 106 (FIG. 4C). The needle 12 may then be axially inserted, with the tip first, into the opening 128 in the proximal end of the first portion 102, and the subcutaneous injection may then be administered. When the injection is complete, the needle spacer 100 and needle 12 may be collectively removed from the skin 20 of the subject, and the luer removal tool 180 (FIG. 4D) may be used to remove the hub 16 of the needle 12 from the needle spacer 100. In particular, the luer removal tool 180 may include an opening that extends to a greater depth than the needle, allowing the removal tool 180 to be seated on the hub 16 with the needle 12 safely located within the opening. The removal tool 180 may then be used to push the hub 16 and needle 12 proximally relative to, and out of the needle spacer 100.

FIGS. 5A-5B illustrate a needle spacer 100 according to another embodiment of the disclosure. As shown, the first portion 102 is configured to engage a hub 16 of a needle 12 that comprises such a hub 16. As shown, the first portion 102 may comprise a hollow, substantially frustoconical portion 134 that tapers from a proximal end thereof to a distal end thereof. The substantially frustoconical shape of first portion 102 may be configured to fit closely, e.g., provide a snap fit over an axially inserted distal end of the hub 16 of the needle 12. A second portion 104 may be coupled to the first portion and may extend distally therefrom. The second portion 104 may include a neck portion 136, which may be substantially tubular, that is coupled to and in communication an interior of the hollow, substantially frustoconical portion 134 at a frustum thereof. The overall shape of first and second portions 102, 104 is that of a funnel. The first portion 102 may be made of a transparent material having a thickness of, e.g., about 0.75 mm.

The neck portion 136 may have an inner diameter that is nominally larger than an outer diameter of the needle 12, such that the neck portion 136 accommodates the needle 12 therein, but does not accommodate the hub 16 of the needle 12 therein. Accordingly, the point of transition from the frustoconically shaped first portion 102 to the tubular neck portion 136 acts as a depth stop, limiting movement of the needle 12 and hub 16 in the distal direction.

The second portion 104, which is depicted in the form of the neck portion 136, may include a skin-contacting surface 108 disposed on a distal end thereof. The skin-contacting surface 108 is configured to engage a surface of skin 20 of a subject. The skin-contacting surface 108 acts as a depth stop during administration, limiting movement of the needle shield 100 in the distal direction when the skin-contacting surface 108 engages the skin 20.

In order to provide additional stability to the needle spacer 100, two or more circumferentially spaced arms 130 may be coupled to the first portion 102, wherein each arm 130 extends radially outwardly and distally relative to the first portion 102. Each arm 130 may include a wing or tab 109 at a distal end thereof, for engaging the skin 20. As shown in FIG. 5B, in certain embodiments, each arm 130 may comprise, at a distal end thereof, a curve 132 in a clockwise or counterclockwise direction about a circumference of the first portion 102, giving the needle spacer an overall shape of spiral galaxy when viewed from the top. As shown in FIG. 5B, four arms 130 are shown, although other numbers may be used, e.g., two arms (FIG. 5A), three arms (FIG. 4C), five arms, or more arms may be provided.

The second portion 104 is configured to maintain a predetermined distance between the hub 16 of the needle 12 and the surface of the skin 20, and to permit insertion of the needle 12 to a depth D of about 4 mm to about 8 mm, e.g., about 6 mm to about 8 mm, about 6 mm, about 7 mm, or about 8 mm below the surface of the skin 20 during administration of a subcutaneous injection. This may be achieved through selection of various dimensions of the needle spacer 100 relative to the length of the needle 12 used, including the length (i.e., height) of the neck portion 136, and the height at which the frustoconical portion 134 transitions to the neck portion 136, thereby engaging and restraining the needle hub 16. In part through the tight fit of the first and second portions 102, 104 around the hub 16 and needle 12, respectively, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection. This visibility may be provided by forming the first and second portions 102, 104 from a transparent material as discussed herein. Additionally, a user may have a relatively unobstructed view through the neck portion 136 between the arms 130.

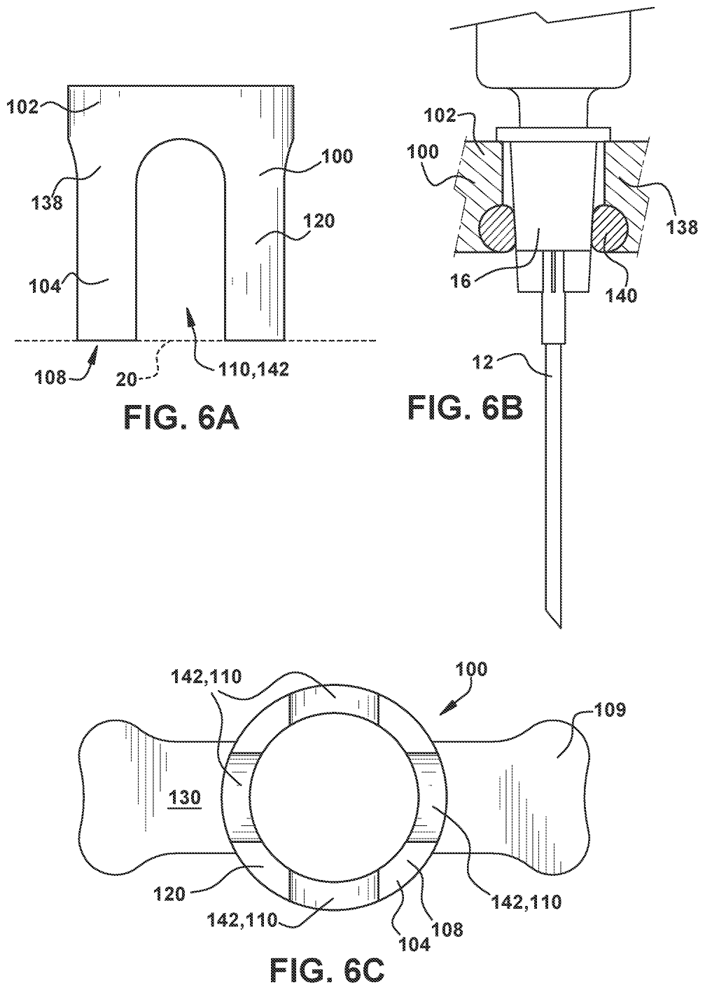

FIGS. 6A-6C illustrate a needle spacer 100 according to another embodiment of the disclosure. As shown, the first portion 102 is configured to engage a hub 16 of a needle 12 that comprises such a hub 16. As shown, the first portion 102 may comprise a hollow cylinder 138 configured to axially receive the needle 12 and hub 16 therein. An annular member 140 (FIG. 6B) may further be disposed between an inner surface of the hollow cylinder 138 and the needle hub 16, to increase friction and engagement between the two features. The hollow cylinder 138 may include an opening 142 at a distal end thereof. The opening 142 may include a cut away portion of the wall of the hollow cylinder 138. In particular, two, three, four, or more openings 142 may be provided about a circumference of the distal end of hollow cylinder 138. FIG. 6C illustrates four openings 142. As shown in FIG. 6A, the opening(s) 142 may extend proximally from the distal end of the hollow cylinder 138 and may be arched in shape.

The portions of the hollow cylinder 138 remaining between the openings 142 may form leg-like projections or legs 120 forming the second portion 104 of the needle spacer 100. These legs 120 extend distally from the first portion 102. FIG. 6C illustrates four such legs 120 forming second portion 104, spaced apart by four openings 142. Each leg 120 may include a skin-contacting surface 108 disposed on a distal end thereof, configured to engage a surface of skin 20 of a subject.

In order to provide additional stability to the needle spacer 100, one or more arms 130 may be coupled the first portion 102. Each arm 130 may include a wing or tab 109 to provide additional surface area connection between the needle spacer 100 and the skin 20. Tape may be used to adhere tabs 109 to the skin 20.

The second portion 104 is configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. For example, the height of the hollow cylinder 138, the height at which the hollow cylinder 138 is configured to engage the hub 16, the length of the needle 12, and other dimensions may be selected in order to arrive at the correct depth of needle insertion. In part through the engagement of the hollow cylinder 138 and the annular member 140 with the hub 16, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection. This may be, for example, through the openings 142, which provide windows 110 through which the injection site may be visualized. Openings 142 may also permit qualitative or quantitative assessment of any leakage at the injection site during or after the administration of the subcutaneous injection.

FIGS. 7A-7C illustrate a needle spacer 100 according to another embodiment of the disclosure. As shown, the needle spacer may be in the form of a cylindrical housing, with first portion 102 forming a proximal wall thereof and having an opening 174 therein that is shaped and dimensioned to engage the hub 16 of the needle. The hub 16 may be inserted axially in a proximal direction into the first portion 102, from the distal end thereof, leading with the hub 16 of the needle 12. The first portion 102 may be adapted to receive the hub 16 while the needle shield 156 (FIG. 7B) remains in place, shielding the tip of the needle 12. In certain embodiments, a pressure sensor 50 is provided, and is coupled to the hub 16 such that the pressure sensor 50 is in fluid connection with the needle 12, similar to the embodiment shown in FIG. 1A. The first portion 102 may retain the pressure sensor in addition to the hub 16 in snap fit engagement. Alternatively, the tubing 14 may be coupled to the proximal end of the hub 16 (as shown in FIG. 1C) in place of the pressure sensor 50 as illustrated in FIGS. 7A-7B.

A second portion 104 may be coupled to the first portion 102 and may extend distally therefrom. The second portion 104 includes axially extending walls of the cylinder, which terminate at a distal end thereof with a skin-contacting surface 108. The skin-contacting surface 108 is configured to engage a surface of skin 20 of a subject. As a result of the axial assembly of the needle and the needle spacer, the skin-contacting surface 108 may extend a full 360 degrees about the needle spacer 100 (FIG. 7C). The skin-contacting surface 108 may further include a wing or tab 109 that extends, e.g., in a radially outward direction from the needle 12 about the entire circumference of the needle spacer 100. The entire skin-contacting surface 108 may include an adhesive disposed on a distal surface thereof, such that constant pressure is applied in 360 degrees around the injection site. In other embodiments, tape may be applied over a proximal face of the skin-contacting surface 108 including tab 109 to adhere the needle spacer 100 to the skin 20.

The second portion 104 is configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. This outcome may be the result of the selection of dimensions including the length of the needle, the height of the second portion 104, the axial point along the hub 16 to which the first portion engages, and other aspects as will be understood by the skilled individual. In part through the engagement of the first portion 102, the hub 16, and optionally, pressure sensor 50, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection, e.g., via windows present in the cylindrical housing. The windows may be of any type previously known or described herein. For example, the windows may be similar to those shown in FIG. 2B, 3, or 4A herein. Such windows permit qualitative or quantitative assessment of any leakage at the injection site during or after the administration of the subcutaneous injection. Alternatively or in addition, the needle spacer 100 including the second portion 104 may be made of a transparent material, which may contribute to the visibility at the injection site.

FIGS. 8A-8E illustrate a needle spacer 100 according to one embodiment of the disclosure. As shown, the first portion 102, shown in FIG. 8B, is configured to laterally engage a hub 16 of a needle 12 that comprises such a hub 16, shown in FIG. 8A. In particular, the first portion 102 comprises two components 102A, 102B (FIGS. 8B, 8E) having complementary geometries and being configured for snap fit engagement with one another about the hub 16 of the needle 12 (FIG. 8A). The first portion 102 may include a cavity 111 (FIGS. 8C, 8E) which may laterally receive and accommodate the hub 16 therein, e.g., in a close-fitting arrangement. In certain embodiments, the components 102A, 102B may be hinged, or may be unitarily formed with a living hinge coupling the components 102A, 102B to one another. The first portion 102 may optionally additionally engage a pressure sensor 50 (FIG. 8D) as described herein.

The foregoing first portion 102 may be combined with features of a number of other embodiments disclosed herein, the specific combinations being omitted solely for brevity. However, a second portion 104 is coupled to the first portion 102 made of 102A, 102B, and extends distally therefrom. As illustrated, the second portion 104 may also extend radially outwardly, such that the needle spacer 100 has a flared or substantially conical shape (as shown in, e.g., FIGS. 8C and 8D). The second portion 104 may include a skin-contacting surface 108 disposed on a distal end thereof. The skin-contacting surface 108 is configured to engage a surface of skin 20 of a subject.

The second portion 104 is configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. The needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use. The needle spacer 100 is further configured to permit visualization of an injection site by a user during the administration of the subcutaneous injection, for example, via transparent material used to form the needle spacer 100.

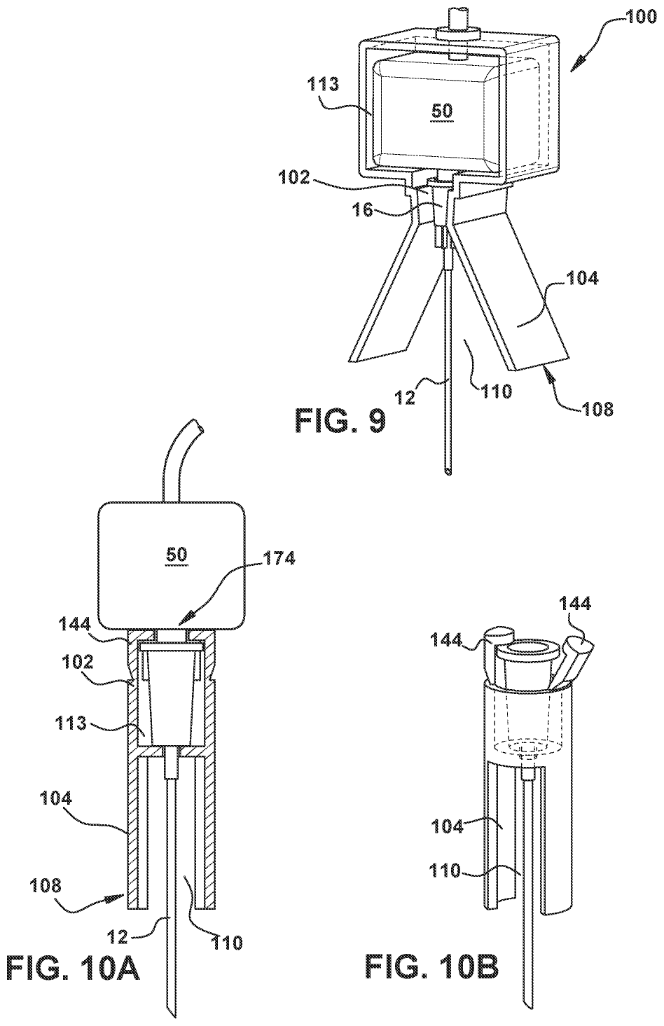

FIG. 9 illustrates a needle spacer 100 according to embodiments of the disclosure. As shown, the first portion 102 is configured to laterally receive and engage a hub 16 of a needle 12 that comprises such a hub 16. The first portion 102 of the needle spacer 100 may further be configured to engage a pressure sensor 50, which may be in fluid connection with the needle 12 as described herein, e.g., relative to FIG. 1A. The first portion 102 may have a snap fit engagement with the pressure sensor 50, and may engage the hub 16 of the needle 12 by conforming to at least a portion of the profile thereof. In particular, the first portion 102 may include a laterally-accessible cavity 113 which is sized and dimensioned to provide a snap fit around pressure sensor 50. As a result of the relatively tighter fit between the pressure sensor 50 and the first portion 102 as compared to the fit between the hub 16 and the first portion 102, the hub 16 and needle 12 may be interchangeable and replaceable in the system 10 (FIG. 1A) without disengaging the first portion 102 from the pressure sensor 50.

A second portion 104 is coupled to the first portion 102 and extends distally therefrom as described herein. The second portion 104 may additionally extend radially outwardly, i.e., the second portion 104 may be flared to provide increased stability. The second portion 104 includes a skin-contacting surface 108 disposed on a distal end thereof, that is configured to engage a surface of skin 20 of a subject. The second portion 104 is also configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. In part through the engagement of the first portion 102 with the hub 16 and the attached pressure sensor 50, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection. In particular, windows or spaces 110 may be provided between solid portions of the second portion 104. These windows 110 may permit qualitative or quantitative assessment of any leakage at the injection site during or after the administration of the subcutaneous injection. Additionally and/or alternatively, the second portion 104 may be made of a transparent material.

FIGS. 10A-10B illustrate a needle spacer 100 according to embodiments of the disclosure. As shown, the first portion 102 includes a laterally accessible cavity 113 that is configured to receive and engage a hub 16 of a needle 12 that comprises such a hub 16. The hub 16 may further be fluidly coupled at a proximal end thereof to a pressure sensor 50 (FIG. 10A), or alternatively, directly to tubing 14 in a manner similar to the embodiments shown in FIGS. 1B-1C. The first portion 102 may substantially enclose a distal end of the hub 16, and substantially but reversibly enclose the proximal end of the hub 16, while accommodating the coupling via proximal opening 174.

The first portion 102 may include a tab(s) 144 disposed on a proximal end thereof, configured to engage a proximal perimeter of the hub 16. In particular, one, two, three, four, or more tabs 144 may be provided. These tabs may be selectively adjusted to either maintain the engagement of the first portion 102 with the hub 16, or to open the proximal end of the first portion 102 sufficiently to enable removal of the hub 16. In particular, the tab(s) may be moved between a first position, in which the tab(s) 144 are retracted to enclose the proximal end of the hub 16 and retain the hub 16 therein, and a second position, in which the tab(s) 144 are extended, and the hub 16 may be freely removed, e.g., in a proximal direction, from the first portion 102.

A second portion 104 is coupled to the first portion 102 and extends distally therefrom. The second portion 104 includes a skin-contacting surface 108 disposed on a distal end thereof, which is configured to engage a surface of skin 20 of a subject. The second portion 104 is also configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. In part through engagement of the first portion 102 with the hub 16, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection, and to permit qualitative or quantitative assessment of any leakage at the injection site during or after the administration of the subcutaneous injection. In particular, windows or spaces 110 may be provided between solid portions of the second portion 104. These windows 110 may permit qualitative or quantitative assessment of any leakage at the injection site during or after the administration of the subcutaneous injection. Additionally and/or alternatively, the second portion 104 may be made of a transparent material.

FIG. 11 illustrates a needle spacer 100 according to one embodiment of the disclosure. As shown, the first portion 102 is configured to engage a hub 16 of a needle 12 that comprises such a hub 16. The first portion 102 may comprise a double-ended luer connector 146, having a male luer lock fitting 148 and a female luer lock fitting 150 on opposing ends. The double-ended luer connector 146 may be oriented such that the male luer lock fitting 148 extends in a distal direction and engages the hub 16 of the needle 12, and the female connection 150 extends in a proximal direction, where it may engage either a pressure sensor 50 or tubing 14. Thus, the needle spacer 100 as shown in FIG. 11 is disposed in use between the needle 12 and the tubing 14 or the pressure sensor 50. The double-ended luer connector 146, the needle hub 16, and the needle 12 are in fluid communication with one another, and the fluid to be subcutaneously injected flows through the needle spacer 100. As a result, the needle spacer 100 as shown in FIG. 11 may be made of a material capable of sterilization.

A second portion 104 is coupled to the first portion 102 and extends distally therefrom. As shown in FIG. 11, the second portion 104 may include an arm(s) 168 extending radially outwardly and distally from the double-ended luer connector, or more particularly, two or more such arms 168. Each arm 168 may include a skin-contacting surface 108 disposed on a distal end thereof. The skin-contacting surface 108 is configured to engage a surface of skin 20 of a subject.

The second portion 104 is configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. This may be achieved by selecting the relative measurements of the length of the needle, the length of the arms 168, the length of the double ended luer connector 146, and the height at which the arms 168 are coupled to the double ended luer connector 146. The needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection. Such visibility may be provided by the spaces or windows 110 between the arms 168 and the needle 12, which provide a user with a clear line of sight to the injection site. These spaces or windows 110 also provide access to permit qualitative or quantitative assessment of any leakage at the injection site during or after the administration of the subcutaneous injection.

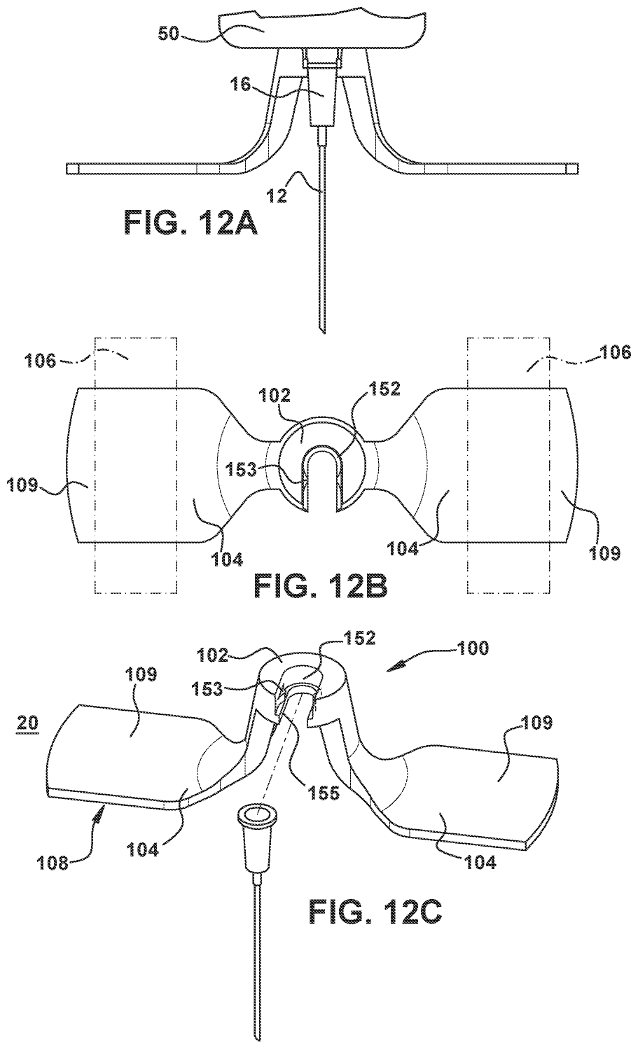

FIGS. 12A-12C illustrate a needle spacer 100 according to one embodiment of the disclosure. As shown, the first portion 102 is configured to engage a hub 16 of a needle 12 that comprises such a hub 16. As discussed elsewhere herein, the hub 16 may be coupled to a pressure sensor 50, and the first portion 102 of the needle spacer may be configured to engage and/or accommodate such pressure sensor 50 (FIG. 12A). As best shown in FIGS. 12B-12C, the first portion 102 may include a snap lock 152 configured to laterally receive and engage the hub 16 of the needle 12. The snap lock 152 may include a cavity within the first portion 102, having an opening to the side, configured for side or lateral entry of the needle hub into the snap lock 152. The wall of the snap lock 152 may be curved and may be substantially C-shaped. Retention features 153 (FIG. 12C) may be provided on an inner surface of the snap lock 152 to contribute to the retention of the hub 16 within the snap lock 152, once inserted. The interior cavity of the snap lock 152 may further include a shoulder or feature 155 which acts as a depth stop, limiting movement of the hub 16 in the distal direction.

A second portion 104 is coupled to the first portion 102 and extends distally therefrom. The second portion 104 includes a skin-contacting surface 108 disposed on a distal end thereof. The skin-contacting surface 108 may include a wing or tab 109. As illustrated in FIGS. 12B-12C, two such wings or tabs 109 are shown, spaced approximately 180 degrees apart, in a butterfly connection arrangement. However, other arrangements and numbers of tabs 109 are equally possible. Through the use of tape 106 or other adhesive as described herein, the wings or tabs 109 may be adhered to the skin 20 of the subject.

In any event, the second portion 104 may extend further in a lateral direction than in a distal direction, meaning that the needle spacer 100 of FIGS. 12B-12C is wider than it is tall. The wings or tabs 109 may be circumferentially offset from the opening to the snap lock 152, so that the wings or tabs 109 do not interfere with the side entry of the hub 16 into the snap lock 152.

The second portion 104 is configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. In particular, the height of the second portion 104, the height of the shoulder 155, the length of the needle 12, and other dimensions may be selected to achieve the desired injection depth.

In part due to the engagement between the snap lock 152 and the hub 16, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection, which may be, e.g., through the use of transparent material to fabricate the needle spacer 100.

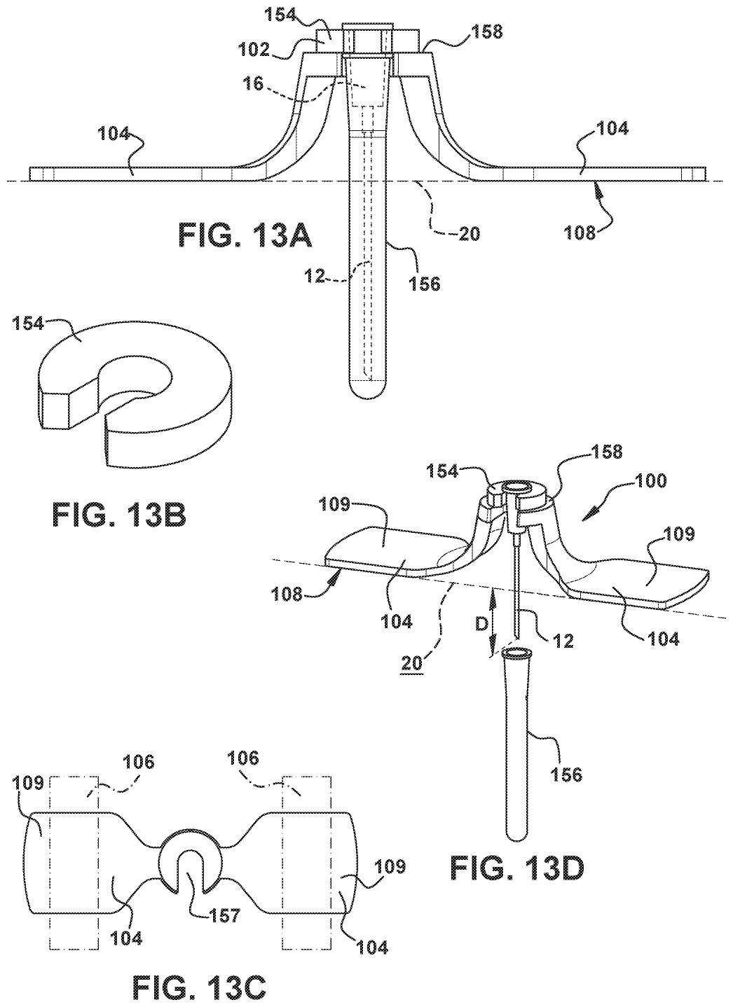

FIGS. 13A-13D illustrate a needle spacer 100 according to another embodiment of the disclosure. As shown, the first portion 102 is configured to engage a hub 16 of a needle 12 that comprises such a hub 16. As best shown in FIGS. 13A-13B, the first portion 102 may include a C-shaped spacer clip 154. Spacer clip 154 may laterally receive the hub 16 therein, e.g., by snapping onto the hub 16 in a direction perpendicular to the longitudinal axis of the needle 12, or rotatably engaging the hub 16 of the needle 12. The spacer clip 154 may be engaged about the hub 16 while the needle shield 156 remains in place shielding the tip of the needle 12. The needle shield 156 may then be removed.

A second portion 104 may be provided, which engages the first portion 102 and extends distally therefrom. The second portion 104 may include a laterally accessible opening 157 defined by a C-shaped wall. The opening 157 may be configured to laterally receive and engage the hub 16 of the needle 12, in a manner similar to the embodiment of FIGS. 12A-12C, while not accommodating the spacer clip 154 therein. The upper surface of the C-shaped wall may further include a proximal shoulder 158, against which the C-shaped spacer clip 154 is configured to bear in use. In cooperation with spacer clip 154, proximal shoulder 158 thus acts as a depth stop, limiting movement of the spacer clip 154, and therefore the engaged hub 16, in the distal direction.

The second portion 104 includes a skin-contacting surface 108 disposed on a distal end thereof. The skin-contacting surface 108 may include a wing or tab 109. As illustrated in FIGS. 13B-13C, two such wings or tabs 109 are shown, spaced approximately 180 degrees apart, in a butterfly connection arrangement. However, other arrangements and numbers of tabs 109 are equally possible. Through the use of tape 106 (FIG. 13C) or other adhesive as described herein, the wings or tabs 109 may be adhered to the skin 20 of the subject.

In any event, the second portion 104, inclusive of tabs or wings 109, may extend further in a lateral direction than in a distal direction, meaning that like the needle spacer 100 of FIGS. 12B-12C, the needle spacer 100 of FIGS. 13A, 13C, and 13D is wider than it is tall. The wings or tabs 109 may be circumferentially offset from the opening 157, so that the wings or tabs 109 do not interfere with the side entry of the hub 16 into the opening 157.

The second portion 104 is configured to maintain a predetermined distance between the hub 16 of the needle 12 and the surface of the skin 20, and to permit insertion of the needle 12 to a depth D of about 4 mm to about 8 mm, e.g., about 6 mm to about 8 mm, about 6 mm, about 7 mm, or about 8 mm below the surface of the skin 20 during administration of a subcutaneous injection. In particular, the height of the second portion 104, and particularly the height of the proximal shoulder 158, the length of the needle 12, the height of the spacer clip 154, and other dimensions may be selected to achieve the desired injection depth.

In part due to the engagement between the opening 157, the spacer clip 154, and the hub 16, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use. The needle spacer 100 is configured to permit visualization of an injection site by a user during the administration of the subcutaneous injection, which may be, e.g., through the use of transparent material to fabricate the needle spacer 100.

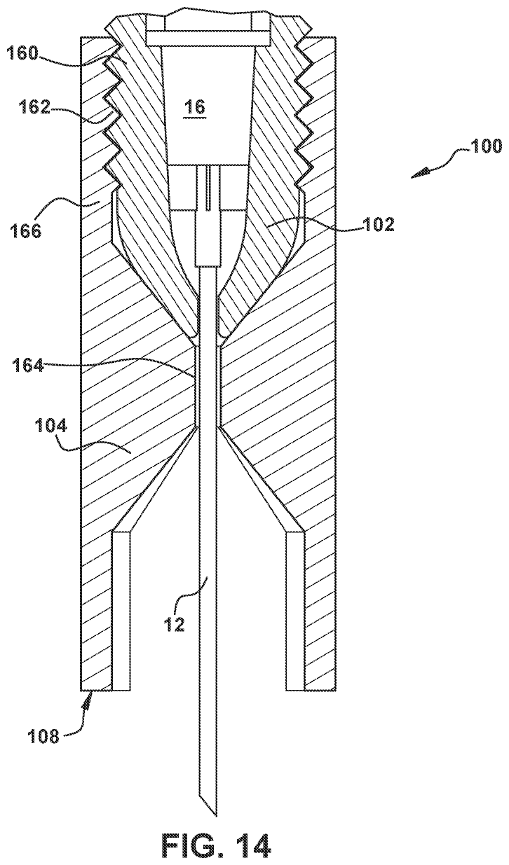

FIG. 14 illustrates a needle spacer 100 according to one embodiment of the disclosure. As shown, the first portion 102 is configured to engage a hub 16 of a needle 12 that comprises such a hub 16. The first portion 102 may comprise a sleeve having external threads 160 disposed thereon. A second portion, comprising a sleeve 166 having internal threads 162 on an inner surface at a proximal end thereof, may be threaded over the first portion 102. In this way, the second portion 104 may be coupled to the first portion 102 by threaded engagement, and the second portion 104 may extend distally relative to the first portion. The second portion may further include an internal shoulder 164 on an inner surface thereof, at a position disposed distally relative to the internal threads 162.

As alluded above, the external threads 160 are configured to threadably engage the internal threads 162 to cause translation of the first portion 102 relative to the second portion 104. As the first portion 102 advances distally relative to the second portion 104, the internal shoulder 164 biases a distal end of the first portion 102 against a radially outer surface of the needle 12, thereby stabilizing the connection, and providing support to the needle 12 in radial, axial, and lateral directions, such that the needle spacer 100 maintains the needle 12 at an approximately 90-degree angle with respect to the surface of the skin 20, and at a predetermined axial position with respect to the needle spacer 100 while in use. The internal shoulder 164 additionally acts as a depth stop, preventing the distal end of the first portion from translating in a distal direction beyond the internal shoulder 164.

The second portion 104 includes a skin-contacting surface 108 disposed on a distal end thereof, that is configured to engage a surface of skin 20 of a subject. The second portion 104 is configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection.

The needle spacer 100 is configured to permit visualization of an injection site by a user during the administration of the subcutaneous injection, for example, by fabricating the needle spacer 100, and particularly the second portion 104 from a transparent material. Additionally or alternatively, windows or openings may be provided in the distal end of the second portion 104, e.g., at a position distal of the internal shoulder 164, similar to windows or openings 110 shown in, e.g., FIGS. 2B, 6A, and 6C.

FIGS. 15A-15B illustrate a needle spacer 100 according to one embodiment of the disclosure. As shown, the first portion 102 is configured to engage a hub 16 of a needle 12 that comprises such a hub 16, which may comprise a female luer fitting. In certain embodiments, the first portion 102 of the needle spacer 100 may further be configured to accommodate and engage a male luer fitting 18 that is coupled to the female luer fitting, such that the entire luer connection is disposed within, and stably retained within the first portion 102. In some embodiments, as shown in FIG. 15A, a pressure sensor 50 may be coupled by a luer fitting to a proximal end of the hub 16. In this embodiment, the first portion 102 of the needle spacer 100 may optionally be configured to additionally engage and accommodate the pressure sensor within the first portion 102. As shown, the first portion 102 comprises a laterally accessible opening 170 sized and dimensioned to accommodate the pressure sensor 50 with a snap fit, or, in the absence of a pressure sensor, the luer connection 16/18 with a snap fit. The size and shape of the laterally accessible opening 170 may vary depending upon the exact configuration. The first portion 102 may also include an opening 174 on a proximal end thereof, configured to allow ingress and egress from the opening 170 of tubing 14 coupled via luer fitting, either directly or indirectly, to the needle 12.

A second portion 104 is coupled to the first portion 102 and extends distally therefrom. As shown in FIGS. 15A-15B, the second portion 104 may essentially be an axial extension of the walls of the first portion 102, and first and second portions 102, 104 may be separated by a retention lip or feature 172. Retention lip 172 defines a distal end of the first portion 102, and acts as a depth stop, limiting the luer fittings 16/18 and/or the pressure sensor 50 from translating in a distal direction.

The distal or bottom end of the second portion 104 may be substantially or entirely open. Due to this openness, as well as the limited depth of the retention lip 172, in some embodiments the needle 12 may be inserted, leading with hub 16, in a proximal direction through the open distal end. Due to the geometries of the first portion 102, the retention lip 172, and the hub 16, the needle 12 may be locked in place via a twist motion, all with the needle shield still in place. As a result, the needle 12 including the hub 16 may be replaced without disengaging the first portion 102 from the pressure sensor 50.

The second portion 104 includes a skin-contacting surface 108 disposed on a distal end thereof, that is configured to engage a surface of skin 20 of a subject. As shown in FIG. 15B, the skin-contacting surface 108 may include a tab or wing 109, which may contribute additional stability to the needle spacer 100 in use. Through the use of tape or other adhesive as described herein, the wings or tabs 109 may be adhered to the skin of the subject.

The second portion 104 is configured to maintain a predetermined distance between the hub 16 of the needle 12 and the surface of the skin 20, and to permit insertion of the needle 12 to a depth of about 4 mm to about 8 mm, e.g., about 6 mm to about 8 mm, about 6 mm, about 7 mm, or about 8 mm below the surface of the skin 20 during administration of a subcutaneous injection. For example, the skilled individual may select dimensions including the length of second portion 104, the length of the needle 12, and the height of the retention lip 172, in order to achieve the desired injection depth. In part due to the snap fit engagement between the first portion 102 and the luer fittings 16/18 and/or the pressure sensor 50, the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

The needle spacer 100 is configured to permit visualization of an injection site by a user during the administration of the subcutaneous injection, for example, by fabricating the needle spacer 100, and particularly the second portion 104 from a transparent material. Additionally or alternatively, windows or openings may be provided in the distal end of the second portion 104, e.g., at a position distal of the retention lip 172, similar to windows or openings 110 shown in, e.g., FIGS. 2B, 6A, and 6C.

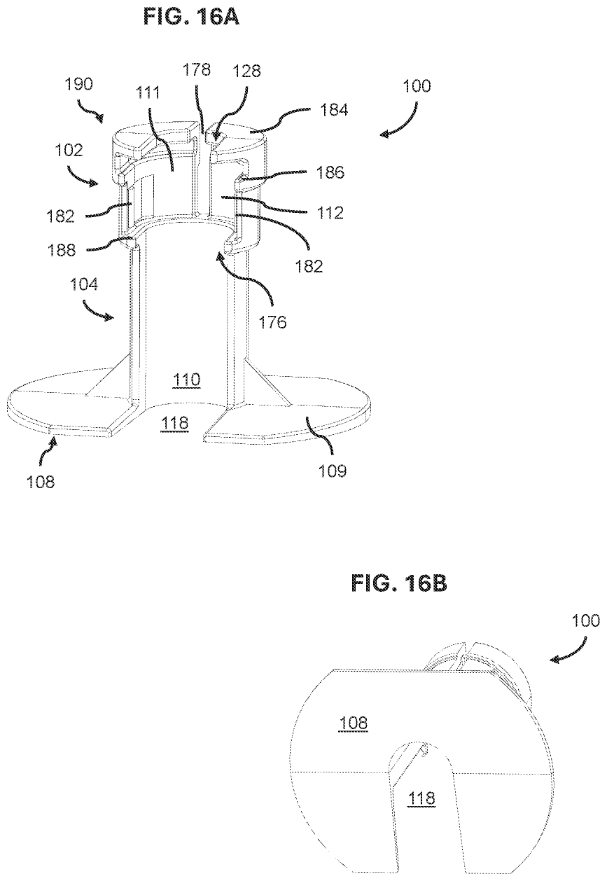

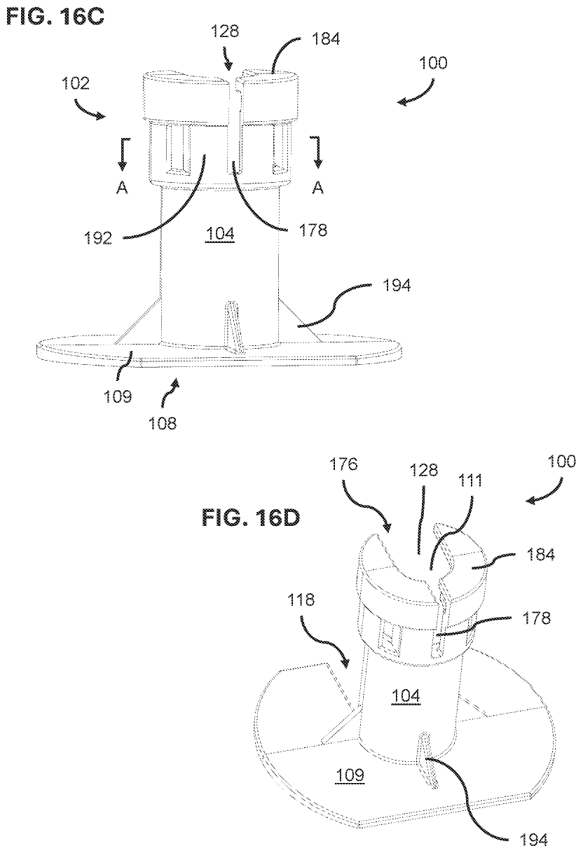

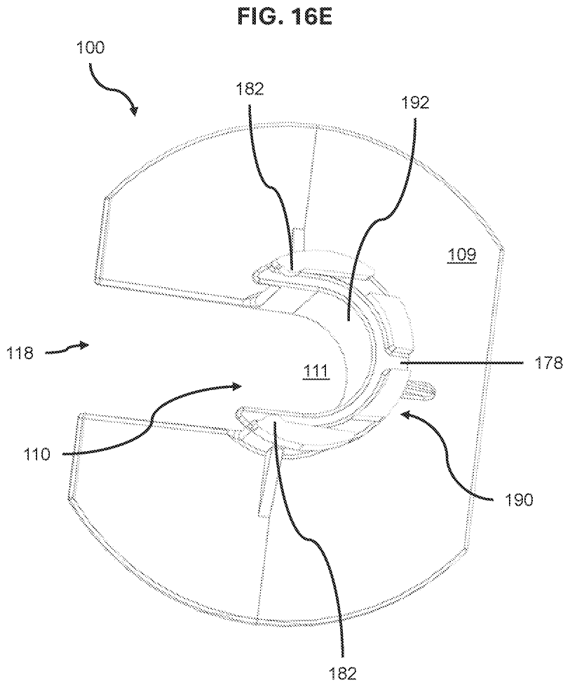

FIGS. 16A-16E illustrate a needle spacer 100 according to one embodiment of the disclosure. As shown, the needle spacer 100 includes a first portion 102 configured to engage a hub of a needle that comprises such a hub, similar to hub 16 of needle 12, described elsewhere herein. The first portion 102 is coupled to a second portion 104, which extends distally from the first portion 102. The second portion 104 is configured to maintain a predetermined distance between the hub of the needle and the surface of the skin, as discussed further below.

With reference to FIG. 16A, the first portion 102 is shaped and dimensioned to provide a housing 190 having a cavity 111 therein, the cavity being adapted to receive and engage the hub of the needle. In certain embodiments, the cavity 111 may particularly be shaped and dimensioned to receive and engage a luer adapter that is coupled to a needle hub. As best shown in FIG. 16E, the cavity 111 may be partially enclosed by a housing side wall 192, having a cross sectional shape that is approximately or substantially circular or semi-circular, although other geometries are also contemplated. Sidewall 192 includes an opening 176 therein, such that the cavity 111 is only partially enclosed. The opening 176 is adapted to provide lateral access to cavity 111, through which the hub and luer adapter may be inserted from the side, i.e. laterally. The laterally accessible opening 176 may extend, e.g., about 180° around the circumference, or up to about 180° around the circumference of the cavity 111. In embodiments in which the cavity 111 has a non-round cross sectional geometry, the laterally accessible opening 176 may extend analogously.

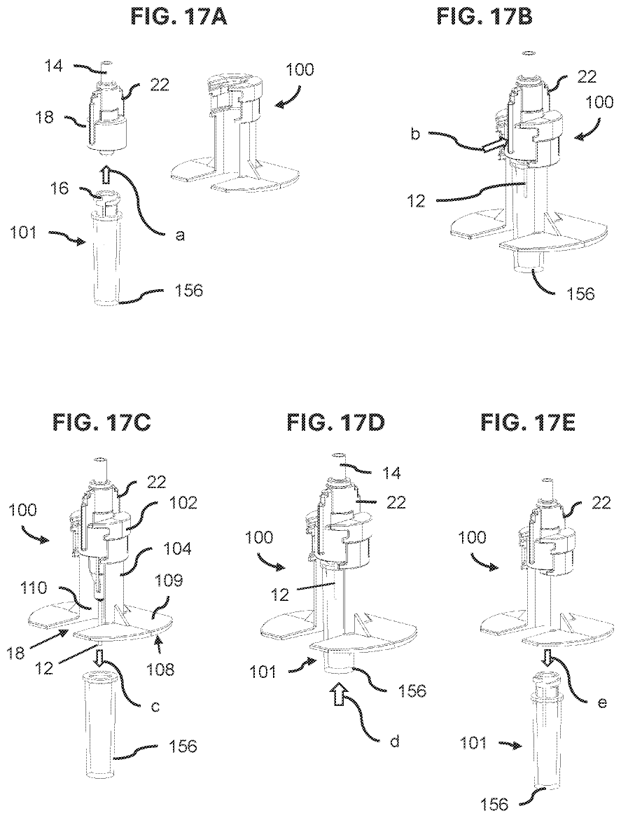

The interior profile 112 of the side walls 192 of the cavity 111 may include a number of features which contribute to the engagement, e.g., snap fit, between the housing 190 and the luer adapter. For example, the cavity 111 may include one or more, e.g., two snap fit features 182, shown in FIGS. 16A and 16E. The snap fit features 182 may be arranged such that one snap fit feature 182 is disposed on each side of the laterally accessible opening 176. The snap fit features 182 may be adapted to engage a body of the luer adapter and to removably secure the luer adapter within the cavity 111 of the housing 190. As shown in FIG. 16E, the snap fit features 182 are adapted to flex radially outward under pressure from the luer adapter to permit insertion into the needle spacer 100 (as shown in FIG. 17B), and to resist radially outward flexion during other phases of use (as shown in, e.g., FIGS. 17C-17E).

With reference to FIGS. 16A, 16C, and 16D, the housing 190 may further include a proximal end 184, which partially encloses the proximal end of the housing 190, thereby constraining the luer adapter disposed within the cavity 111 from translating in a proximal direction relative to the housing 190. The proximal end 184 may include an opening 128 extending through a thickness thereof, through which a portion of the luer adapter and/or tubing (such as tubing 14 in FIG. 1B and others) connected thereto, may extend in a proximal direction (as shown in, e.g., FIGS. 17B-17E). In certain embodiments, the opening 128 may also accommodate fluid connection between the needle, the hub, and a pressure sensor such as pressure sensor 50 (FIG. 1A, 1B, and others). The opening 128 may also extend radially outward in one or more directions, placing the opening 128 in continuous communication with the laterally accessible opening 176, and facilitating lateral insertion of the features described above into opening 128, via laterally accessible opening 176.

As shown in FIG. 16A, the housing 190 may further include a first retention lip 186 disposed on the interior profile 112 of the cavity 111. The first retention lip 186 may be adapted to retain a lip on a proximal end of the luer adapter. Such a lip on the luer adapter may be laterally inserted (i.e. inserted from the side) through laterally accessible opening 176, and matingly received between the proximal end 184 and the first retention lip 186. The luer adapter lip may be held securely in place such that the fit constrains distal or proximal translation of the luer adapter and attached needle relative to the housing 190. A second retention lip 188 may be disposed on the interior profile 112 of the cavity 111 at the distal end thereof, and may be adapted to constrain the luer adapter and attached needle from translating in a distal direction relative to the housing 190.

As best shown in FIG. 16C, the housing 190 may further include a relief cut 178 in a side wall 192 thereof. The relief cut 178 may be disposed, e.g., on an opposite side of the housing 190 from the laterally accessible opening 176. As shown, the relief cut 178 may extend through a full thickness of the side wall of the housing 190, although in other embodiments the relief cut may extend through partial thickness of side wall 192. The relief cut 178 may be shaped and dimensioned to accommodate and receive a radially-outward extending rib disposed on a luer adapter, such as rib 22 on luer adapter 18 as shown in, e.g., FIGS. 17B-17E. The engagement of the rib with the relief cut 178 constrains rotation of the luer adapter, hub, and associated needle relative to the housing 190, and assists with the snap fit engagement. As shown in FIGS. 16A, 16C, and 16D, the relief cut 178 may extend distally from the proximal end 184 through the housing 190 side wall 192, e.g., to a depth of the second retention lip 188. Thus, in certain embodiments, the proximal-facing shoulder surface created by the second retention lip 188 may be co-planar or approximately co-planar with a bottom or distal-most surface of the relief cut 178. The relief cut 178 may further extend radially from the opening 128 to the relief cut 178, placing these features in fluid communication with one another. This allows a luer adapter with associated hub and needle to be inserted from the side into laterally accessible opening 176, and continuing the insertion until the rib is seated in the relief cut 178 opposite the laterally accessible opening 176, and the luer adapter is seated in the cavity 111.

With reference to FIGS. 16A-16D, and as discussed above, the first portion 102, which may be in the form of housing 190, is coupled to a second portion 104 which extends distally therefrom. The second portion 104 may be partially enclosed, and may have a cross sectional shape that is similar to that of the first portion 102, as it may be formed as a distal extension thereof. In certain examples, the second portion may have a cross sectional shape that is approximately or substantially circular or semi-circular, although other geometries are also contemplated. The second portion 104 includes an opening or window 110 therein, such that the second portion 104 is only partially enclosed. The window 110 is adapted to provide lateral access to the interior of the second portion to, e.g., the needle and a needle shield disposed over the needle, as well as visualization as discussed further herein. The window 110 may extend, e.g., about 180° around the circumference, or up to about 180° around the circumference of the second portion 104. In embodiments in which the second portion has a non-round cross sectional geometry, the window 110 may extend analogously.

The second portion 104 may have an inner dimension, e.g., inner diameter, that is sufficiently large to accommodate not only the needle, but also an associated needle shield (such as needle shield 156, FIGS. 17A-17E) coupled to the hub and covering the needle. The window 110 is aligned and continuous with the laterally accessible opening 176 in the housing 190, and an opening 118 in the tab 109 (discussed below). Collectively, the laterally accessible opening 176 in the housing 190, the window or opening 110 in the second portion 104, and the opening 118 in the tab 109 allow the entire needle assembly, including the needle, needle shield, hub, and an attached luer adapter to be laterally inserted into the needle spacer. The needle spacer 100 is further configured to permit visualization of the injection site by a user during the administration of the subcutaneous injection, e.g., through windows 110. The window 110 may be, e.g., an opening in the material from which the second portion 104 is constructed. Window 110 further provides access to the injection site to permit qualitative or quantitative assessment of any leakage at the injection site during or after the administration of the subcutaneous injection, as well as monitoring for swelling, bruising, redness, or other adverse events.

The second portion 104 may include a skin-contacting surface 108 disposed on a distal end thereof. The skin contacting surface 108 may further include one or more wings or tabs 109. As illustrated, one tab 109 extends radially outwardly from the second portion 104, excepting the opening 118 in the tab 109 which is aligned with, and in continuous communication with window 110 in second portion 104 and laterally accessible opening 176 in first portion 102. Tab 109 is shaped and dimensioned to facilitate holding the needle spacer 100 in place at the injection site with fingers placed on the tab 109. Tab 109 also increases the contacting surface area between the needle spacer 100 and the skin 20, thereby increasing the stability of the needle spacer 100. This stability may be aided in certain embodiments by support members 194 (FIG. 16C), which assist in maintaining the orientation of the needle spacer 100 relative to the wing or tab 109 and the skin of the subject. In certain embodiments, one, two, three, or more support members 194 may be positioned about the interface between the second portion 104 and the tab 109 (three are shown in FIGS. 16C, 16D). If desired, tab 109 also provides a point of contact wherein tape may be applied over the tab 109 and the subject's skin to adhere the needle spacer 100 to the skin, although the needle spacer 100 is adapted for use in the absence of any adhesive.

The second portion 104 is configured to maintain a predetermined distance between the hub of the needle and the surface of the skin, and to permit insertion of the needle 12 to a depth of about 4 mm to about 8 mm, e.g., about 6 mm to about 8 mm, about 6 mm, about 7 mm, or about 8 mm below the surface of the skin during administration of a subcutaneous injection. In particular, the depth to which the needle may be inserted is a function of the needle length and certain dimensions of the needle spacer 100 which may be selected accordingly. For example, in addition to needle length, the depth to which the needle may be inserted may be a function of the distance from the second retention lip 188 to the skin-contacting surface 108 (e.g., in millimeters), e.g., the height of the second portion 104. In part through the engagement of the first portion 102, e.g., the housing 190 with the hub and/or luer adapter, the needle spacer 100 is configured to support the needle radially, axially, and laterally, such that the needle spacer 100 maintains the needle at an approximately 90-degree angle with respect to the surface of the skin, and at a predetermined axial position with respect to the needle spacer 100 while in use.

Turning next to FIGS. 17A-17E, these figures illustrate steps in the assembly of the needle spacer 100 as shown in the embodiment of FIGS. 16A-16E.

As shown in FIG. 17A, a needle spacer 100, a luer adapter 18, and a needle assembly 101 are provided. The needle assembly 101 may include a needle (such as needle 12, FIG. 17B) which may be covered by a needle shield 156, and may be coupled to a hub 16. As indicated by the arrow “a,” the needle assembly 101, via hub 16, may be coupled to a distal end of the luer adapter 18. The luer adapter 18 may further be coupled to tubing 14 at a proximal end thereof as described relative to, e.g., FIG. 1B. The luer adapter 18 may include one or more ribs 22 extending radially outward therefrom. In the embodiment depicted in FIGS. 17A-17E, the luer adapter 18 includes two ribs 22 extending radially outward therefrom, equally or approximately equally spaced with respect to one another about the circumference of the luer adapter 18. In other embodiments, a luer adapter having other numbers of ribs 22, e.g., three ribs 22, and needle spacers 100 having corresponding and mating features (e.g. corresponding relief cuts and/or openings), are also contemplated.

As shown in FIG. 17B, the needle assembly may be laterally inserted, as shown by the direction of the arrow “b”, into the cavity 111 in the first portion 102 of the needle spacer 100. In particular, the luer adapter 18 may be received within the cavity 111, and may be retained securely in place within the cavity 111 by snap fit features 182 (best shown in FIGS. 16A and 16E) on the interior profile 112 of the cavity 111. When fully inserted, one rib 22 may be received within a relief cut 178 (best shown in FIG. 16C) in the housing 190 of the first portion 102 of the needle spacer, contributing to the snap fit engagement. Another rib 22 may be located within the laterally accessible opening 176. The second portion 104 of the needle spacer may have an inner dimension sufficiently large to laterally receive the needle shield 156 within the second portion 104, with the needle shield further being permitted to be inserted laterally through the opening 118 in the tab 109.

As shown in FIG. 17C, the needle shield 156 may be removed from the needle 12, as shown by the direction of arrow “c”. The needle shield 100 may then be placed on the injection site on the subject, with the skin-contacting surface 108 in contact with the subject. The needle spacer 100 may be held in place, e.g., using fingers, tape, adhesive, etc. The subcutaneous injection may then be performed, with bolus visibility through the window 110 in the second portion 104.

As shown in FIG. 17D, after completion of the injection, the needle spacer 100, including the engaged needle assembly 101 and luer adapter 18 may be removed from the injection site, and the needle shield 156 may be replaced over the needle 12 as shown by the direction of arrow “d”.

As shown in FIG. 17E, the needle assembly may then be removed from the luer adapter 18 and the needle spacer 100 as shown by the direction of arrow “e”, and disposed in, e.g., a sharps container.

Embodiments of the present disclosure may include the following features:

Item 1. A needle spacer 100 comprising: a first portion 102 configured to engage a hub 16 of a needle 12 comprising the hub 16; and a second portion 104 coupled to the first portion 102 and extending distally therefrom, the second portion 104 including a skin-contacting surface 108 disposed on a distal end thereof, wherein the skin-contacting surface 108 is configured to engage a surface of skin 20 of a subject, and wherein the second portion 104 is configured to maintain a predetermined distance between the hub 16 of the needle 12 and the surface of the skin 20, and to permit insertion of the needle 12 to a depth of about 4 mm to about 8 mm below the surface of the skin 20 during administration of a subcutaneous injection via the needle 12.

Item 2. The needle spacer 100 of item 1, wherein the needle spacer 100 is configured to support the needle 12 radially, axially, and laterally, such that the needle spacer 100 maintains the needle 12 at an approximately 90-degree angle with respect to the surface of the skin 20, and at a predetermined axial position with respect to the needle spacer 100 while in use.

Item 3. The needle spacer of item 1, wherein the needle spacer 100 is configured to permit visualization of an injection site by a user during the administration of the subcutaneous injection.

Item 4. The needle spacer of item 3, wherein the needle spacer 100 is configured to permit qualitative or quantitative assessment of any leakage at the injection site during or after the administration of the subcutaneous injection.

Item 5. The needle spacer of item 3, wherein one or both of the first portion or the second portion comprise a transparent material.

Item 6. The needle spacer of item 3, wherein the second portion comprises an opening or a window through which the injection site is visible to the user during the administration of the subcutaneous injection.

Item 7. The needle spacer of any one of items 1 to 6, wherein the first portion 102 of the needle spacer 100 is configured to laterally receive the hub 16 of the needle 12.

Item 8. The needle spacer of any one of items 1 to 6, wherein the first portion 102 of the needle spacer 100 is configured to axially receive the hub 16 of the needle 12.

Item 9. The needle spacer of item 1, wherein the skin-contacting surface 108 further comprises a laterally extending wing or tab 109.

Item 10. The needle spacer of item 9, wherein the laterally extending wing or tab 109 further comprises a plurality of laterally extending wings or tabs 109.

Item 11. The needle spacer of item 9, wherein the laterally extending wing or tab 109 is configured to receive tape 106 on a proximal face thereof, and wherein the tape 106 adheres the laterally extending wing or tab 109 to the skin 20.

Item 12. The needle spacer of item 1, wherein the skin-contacting surface 108 comprises an adhesive disposed on a distal surface thereof.

Item 13. The needle spacer of any one of item 1-6, wherein the first portion 102 of the needle spacer is further configured to engage a pressure sensor 50, and wherein the pressure sensor 50 is in fluid connection with the needle 12.

Item 14. The needle spacer of item 8, wherein the first portion 102 comprises a cavity 111 therein open to a proximal end of the first portion 102, and the cavity 111 comprises an interior profile 112 configured to complement and to matingly receive a distal end of the needle hub 16.

Item 15. The needle spacer of item 14, further comprising a neck portion 114 coupling the first portion 102 to the second portion 104, wherein the neck portion 114 comprises an inner diameter that is greater than an outer diameter of the needle 12, and smaller than an outer diameter of the needle hub 16, such that the neck portion acts as a depth stop limiting movement of the needle hub 16 in a distal direction, and wherein the second portion 104 comprises a concave geometry that is open to a distal end of the needle spacer 100.

Item 16. The needle spacer of item 7, wherein: the first portion 102 comprises a substantially annular shape having a first opening 116 therein, the first opening 116 being configured to laterally receive the hub 16 of the needle 12; and the second portion 104 comprises a leg member 120 extending distally and radially outwardly relative to the first portion 102 and a base member 122 coupled to the leg member 120, the base member 122 comprises a substantially annular shape having a second opening 118 therein, wherein the second opening 118 is configured to laterally receive the needle therein, and is circumferentially aligned with the first opening 116, and a diameter of the base member 122 is greater than a diameter of the first portion 102.

Item 17. The needle spacer of item 16, wherein the leg member 120 comprises a plurality of leg members 120, and each leg member 120 of the plurality of leg members 120 is circumferentially spaced about the first portion 102 and the base member 122 relative to each other leg member 120.

Item 18. The needle spacer of item 8, further comprising: an opening 128 in a proximal end of the first portion 102, configured to receive the hub 16 of the needle 12 therein; and a plurality of snap arms 124 disposed about the opening 128, wherein the plurality of snap arms 124 are configured to collectively engage the hub 16 of the needle 12.

Item 19. The needle spacer of item 8, further comprising: an opening 128 in a proximal end of the first portion 102, configured to receive the hub 16 of the needle 12 therein; and a friction member 126 disposed about a perimeter of the opening 128, wherein the friction member 126 is configured to engage the hub 16 of the needle 12, and the friction member 126 is selected from a high friction coating and an O-ring.

Item 20. The needle spacer of item 8, further comprising: a plurality of circumferentially spaced arms 130 coupled to the first portion 102, wherein each arm 130 of the plurality of arms extends radially outwardly and distally relative to the first portion 102, and each arm 130 includes a skin-contacting surface 108 at a distal end thereof, and wherein the plurality of arms 130 collectively form a domed shape, thereby maintaining the predetermined distance between the hub 16 of the needle 12 and the surface of the skin 20 during the administration of the subcutaneous injection.

Item 21. The needle spacer of item 20, wherein each arm 130 in the plurality of arms 130 comprises, at a distal end thereof, a curve 132 about a circumference of the first portion 102.

Item 22. The needle spacer of item 8, wherein the first portion 102 comprises a hollow, substantially frustoconical portion 134 tapering from a proximal end thereof to a distal end thereof, the first portion 102 being configured to fit closely over a distal end of the hub 16 of the needle 12, and wherein the second portion comprises 104 a neck portion 136 coupled to, and in communication an interior of the hollow, substantially frustoconical portion 134 at a frustum thereof, the neck portion 136 having an inner diameter that is nominally larger than an outer diameter of the needle, such that the neck portion accommodates the needle 12 therein, but does not accommodate the hub 16 of the needle 12 therein.

Item 23. The needle spacer of item 8, wherein the first portion 102 comprises: a hollow cylinder 138 having an opening 142 in a distal end thereof; and an annular member 140 disposed between the hollow cylinder 138 and the needle hub 16.

Item 24. The needle spacer of item 8, wherein the skin-contacting surface 108 extends 360 degrees about the needle 12.

Item 25. The needle spacer of item 7, wherein the first portion 102 comprises two components having complementary geometries and being configured for snap fit engagement with one another about the hub 16 of the needle 12.

Item 26. The needle spacer of item 7, wherein the first portion 102 is further configured to engage a pressure sensor 50 disposed proximally of the hub 16 of the needle 12, such that the hub 16 of the needle 12 is replaceable without disengaging the first portion 102 from the pressure sensor 50.

Item 27. The needle spacer of item 7, wherein the first portion 102 comprises a tab 144 configured to engage a proximal end of the hub 16 of the needle 12.

Item 28. The needle spacer of item 8, wherein the first portion 102 comprises a double-ended luer connector, having a male luer lock fitting 148 and a female luer lock fitting 150 on opposing ends, wherein the male luer lock fitting 148 extends in a distal direction and engages the hub 16 of the needle 12, and the female connection 150 extends in a proximal direction, and the double-ended luer connector 146, the needle hub 16, and the needle 12 are in fluid communication with one another.

Item 29. The needle spacer of item 7, wherein the first portion 102 comprises a snap lock configured to laterally receive and engage the hub 16 of the needle 12, and wherein the second portion 104 extends further in a lateral direction than in a distal direction.

Item 30. The needle spacer of item 7, wherein the first portion 102 comprises a C-shaped spacer clip 154 configured to engage the hub 16 of the needle 12.

Item 31. The needle spacer of item 30, wherein the second portion 104 comprises a proximal shoulder, against which the C-shaped spacer clip 154 is configured to bear in use.

Item 32. The needle spacer of item 8, wherein the first portion 102 comprises external threads 160; the second portion 104 comprises a sleeve 166 disposed over the first portion 102, the sleeve 166 having internal threads 162 on an inner surface thereof, and an internal shoulder 164 disposed distally relative to the internal threads 162, wherein the external threads 160 are configured to threadably engage the internal threads 162 to cause translation of the first portion 102 relative to the second portion 104; and as the first portion 102 advances distally relative to the second portion 104, the internal shoulder 164 biases a distal end of the first portion 102 against a radially outer surface of the needle 12.

Item 33. The needle spacer of item 7, wherein the hub 16 of the needle 12 comprises a female luer fitting, and the first portion 102 of the needle spacer 100 is further configured to engage a male luer fitting 18 that is coupled to the female luer fitting.

Item 34. The needle spacer 100 of item 7 or item 33, wherein the skin contacting surface 108 further comprises a laterally extending wing or tab 109.

Item 35. The needle spacer 100 of item 33, wherein the first portion 102 comprises a housing 190 having a cavity 111 therein, and a laterally accessible opening 176 to the cavity 111, wherein the laterally accessible opening 176 and the cavity 111 are adapted to matingly receive the male luer fitting 18.

Item 36. The needle spacer 100 of item 35, wherein the window 110 in the second portion 104 is in fluid communication with the laterally accessible opening 176 in the first portion 102, such that as the male luer fitting 18 is laterally received in the cavity 111, the window 110 is adapted to laterally receive the needle 12 and a needle shield 156 disposed over the needle 12.

Item 37. The needle spacer 100 of item 35, further comprising a plurality of snap fit features 182 disposed on each side of the laterally accessible opening 176, wherein the plurality of snap fit features 182 are adapted to flex radially outwardly to allow insertion of the male luer fitting 18, and to retain the male luer fitting 18 within the cavity 111.

Item 38. The needle spacer 100 of item 35, further comprising a relief cut 178 in a side wall 192 of the housing 190, disposed opposite the laterally accessible opening 176, wherein the relief cut 178 is adapted to receive a rib 22 on the male luer fitting 18.

Item 39. A system 10 comprising: a needle 12 coupled to a hub 16 and configured for subcutaneous injection into skin 20 of a subject; tubing 14 coupled to, and in fluid communication with the hub 16 and the needle 12; and a needle spacer 100 disposed between, and configured to maintain a predetermined distance between the hub 16 of the needle 12 and the skin 20 of the subject, the needle spacer 100 comprising: a first portion 102 configured to engage the hub 16 of the needle 12; and a second portion 104 coupled to the first portion 102 and extending distally therefrom, the second portion 104 including a skin-contacting surface 108 disposed on a distal end thereof, wherein the skin-contacting surface 108 is configured to engage the surface of the skin 20, and wherein the second portion 104 is configured to permit insertion of the needle 12 to a depth of about 4 mm to about 8 mm into the skin 20 during administration of a subcutaneous injection via the needle 12.

Item 40. The system of item 39, further comprising: a syringe 30 fluidly coupled to a proximal end of the tubing 14; and an infusion pump 40 configured to administer a fluid contained in the syringe via subcutaneous injection.

Item 41. The system of item 40, wherein the fluid is selected from a medicament, a solution, a suspension, saline, or hyaluronic acid.

Item 42. The system of item 39, wherein the needle 12 has a length of about 13 mm or greater.

Item 43. The system of item 39, wherein a distal end of the tubing 14 is coupled to the needle hub 16 by complementary luer fittings.

Item 44. The system of item 43, wherein the complementary luer fittings are luer lock fittings.

Item 45. The system of item 43, wherein the needle spacer 100 is configured so as to permit unimpeded access to the luer fittings in use.

Item 46. The system of item 39, further comprising a pressure sensor 50 in fluid connection with the needle 12, the hub 16, and the tubing 14, wherein the first portion 102 of the needle spacer 100 is configured to engage the pressure sensor 50.

What is claimed is:

1. A needle spacer comprising:
a first portion configured to engage a hub of a needle comprising the hub; and
a second portion coupled to the first portion and extending distally therefrom, the second portion including a skin-contacting surface disposed on a distal end thereof, and configured to engage a surface of skin of a subject, and
wherein the second portion is configured to maintain a predetermined distance between the hub of the needle and the surface of the skin, and to permit insertion of the needle to a depth of about 4 mm to about 8 mm below the surface of the skin during administration of a subcutaneous injection via the needle.

2. The needle spacer of claim 1,
wherein the needle spacer is configured to support the needle radially, axially, and laterally, such that the needle spacer maintains the needle at an approximately 90-degree angle with respect to the surface of the skin, and at a predetermined axial position with respect to the needle spacer while in use, and
wherein the needle spacer is configured to permit visualization of an injection site by a user during the administration of the subcutaneous injection, and qualitative or quantitative assessment of any leakage at the injection site during or after the administration of the subcutaneous injection.

3. The needle spacer of claim 2, wherein one or both of the first portion or the second portion comprise a transparent material, or wherein the second portion comprises an opening or a window through which the injection site is visible to the user during the administration of the subcutaneous injection.

4. The needle spacer of claim 1, wherein the skin-contacting surface further comprises a laterally extending wing or tab.

5. The needle spacer of claim 1, wherein the first portion of the needle spacer is further configured to engage a pressure sensor that is fluidly coupled to the needle, wherein the pressure sensor is disposed proximally of the hub of the needle.

6. The needle spacer of claim 1, further comprising:
a neck portion coupling the first portion to the second portion; and
a cavity disposed within the first portion, wherein the cavity is open to a proximal end of the first portion, and includes an interior profile configured to complement and to matingly receive an axially inserted distal end of the needle hub,
wherein the neck portion comprises an inner diameter that is greater than an outer diameter of the needle, and smaller than an outer diameter of the needle hub, such that the neck portion acts as a depth stop limiting movement of the needle hub in a distal direction, and
wherein the second portion comprises a concave geometry that is open to a distal end of the needle spacer.

7. The needle spacer of claim 1, wherein:
the first portion comprises a substantially annular shape having a first opening therein, the first opening being configured to laterally receive the hub of the needle; and
the second portion comprises a leg member extending distally and radially outwardly relative to the first portion and a base member coupled to the leg member,
the base member comprises a substantially annular shape having a second opening therein, wherein the second opening is configured to laterally receive the needle therein, and is circumferentially aligned with the first opening, and
a diameter of the base member is greater than a diameter of the first portion.

8. The needle spacer of claim 1, further comprising:
an opening in a proximal end of the first portion, configured to axially receive the hub of the needle therein;
a friction member disposed about a perimeter of the opening, wherein the friction member is configured to engage the hub of the needle, and the friction member is selected from a high friction coating and an O-ring; and
a plurality of snap arms disposed about the opening, wherein the plurality of snap arms are configured to collectively engage the hub of the needle.

9. The needle spacer of claim 1, further comprising:
a plurality of circumferentially spaced arms coupled to the first portion, wherein each arm of the plurality of arms extends radially outwardly and distally relative to the first portion, and each arm includes a skin-contacting surface at a distal end thereof, and
wherein the plurality of arms collectively form a domed shape, thereby maintaining the predetermined distance between the hub of the needle and the surface of the skin during the administration of the subcutaneous injection, and
wherein each arm in the plurality of arms comprises, at a distal end thereof, a curve about a circumference of the first portion, and
wherein the first portion of the needle spacer is configured to axially receive the hub of the needle.

10. The needle spacer of claim 1, wherein the first portion comprises a hollow, substantially frustoconical portion tapering from a proximal end thereof to a distal end thereof, the first portion being configured to axially receive, and fit closely over a distal end of the hub of the needle, and
wherein the second portion comprises a neck portion coupled to, and in communication an interior of the hollow, substantially frustoconical portion at a frustum thereof, the neck portion having an inner diameter that is nominally larger than an outer diameter of the needle, such that the neck portion accommodates the needle therein, but does not accommodate the hub of the needle therein.

11. The needle spacer of claim 1, wherein the first portion of the needle spacer is configured to axially receive the hub of the needle, and comprises:
a hollow cylinder having an opening in a distal end thereof; and
an annular member disposed between an inner surface of the hollow cylinder and the needle hub.

12. The needle spacer of claim 1, wherein the first portion comprises two components having complementary geometries and being configured for snap fit engagement with one another about the hub of the needle,
wherein the first portion of the needle spacer is configured to laterally receive the hub of the needle.

13. The needle spacer of claim 1, wherein the first portion comprises a double-ended luer connector, having a male luer lock fitting and a female luer lock fitting on opposing ends,
wherein the male luer lock fitting extends in a distal direction and is configured to axially engage the hub of the needle, and the female luer lock fitting extends in a proximal direction, and
the double-ended luer connector, the needle hub, and the needle are in fluid communication with one another.

14. The needle spacer of claim 1, wherein the first portion comprises a snap lock or a tab configured to laterally receive and engage the hub of the needle, and wherein the second portion extends further in a lateral direction than in a distal direction.

15. The needle spacer of claim 1, wherein the first portion comprises a C-shaped spacer clip configured to laterally receive and engage the hub of the needle, and
wherein the second portion comprises a proximal shoulder, against which the C-shaped spacer clip is configured to bear in use.

16. The needle spacer of claim 1, wherein the first portion comprises external threads and is configured to axially receive the hub of the needle;
the second portion comprises a sleeve disposed over the first portion, the sleeve having internal threads on an inner surface thereof, and an internal shoulder disposed distally relative to the internal threads,
wherein the external threads are configured to threadably engage the internal threads to cause translation of the first portion relative to the second portion; and as the first portion advances distally relative to the second portion, the internal shoulder biases a distal end of the first portion against a radially outer surface of the needle.

17. The needle spacer of claim 1, wherein the first portion comprises a housing having a cavity therein, and a laterally accessible opening to the cavity,
the hub of the needle comprises a female luer fitting, and a male luer fitting is coupled to the female luer fitting,
the cavity of the housing is configured to laterally receive and engage the male luer fitting through the laterally accessible opening.

18. The needle spacer of claim 17, further comprising a window in the second portion, through which the injection site is visible to the user during the administration of the subcutaneous injection,
wherein the window is in fluid communication with the laterally accessible opening in the first portion, such that as the male luer fitting is laterally received in the cavity, the window is adapted to laterally receive the needle and a needle shield disposed over the needle.

19. The needle spacer of claim 17, further comprising:
a plurality of snap fit features disposed on each side of the laterally accessible opening, wherein each snap fit feature of the plurality of snap fit features is adapted to flex radially outwardly to allow insertion of the male luer fitting, and to retain the male luer fitting within the cavity; and
a relief cut in a side wall of the housing, disposed opposite the laterally accessible opening, wherein the relief cut is adapted to receive a rib on the male luer fitting.

20. A system comprising:
a needle coupled to a hub and configured for subcutaneous injection into skin of a subject;
tubing coupled to, and in fluid communication with the hub and the needle; and
a needle spacer disposed between, and configured to maintain a predetermined distance between the hub of the needle and the skin of the subject, the needle spacer comprising:
a first portion configured to engage the hub of the needle; and
a second portion coupled to the first portion and extending distally therefrom, the second portion including a skin-contacting surface disposed on a distal end thereof, and configured to engage the surface of the skin, and
wherein the second portion is configured to permit insertion of the needle to a depth of about 4 mm to about 8 mm into the skin during administration of a subcutaneous injection via the needle.
